(19) United States
(12) Patent Application Publication
(10) Pub. No.: US 2018/0140679 A1
(43) Pub. Date: May 24, 2018

(54) MODULATION OF AXL RECEPTOR ACTIVITY IN COMBINATION WITH CYTOREDUCTIVE THERAPY

(71) Applicant: THE BOARD OF TRUSTEES OF THE LELAND STANFORD JUNIOR UNIVERSITY, STANFORD, CA (US)

(72) Inventors: Amato J. Giaccia, Stanford, CA (US); Jennifer R. Cochran, Stanford, CA (US); Yu Miao, Sunnyvale, CA (US); Mihalis Kariolis, San Mateo, CA (US)

(21) Appl. No.: 15/818,251

(22) Filed: Nov. 20, 2017

Related U.S. Application Data
(60) Provisional application No. 62/426,016, filed on Nov. 23, 2016.

(51) Int. Cl. A61K 38/45; A61K 45/06; A61K 31/704

(52) U.S. Cl. CPC A61K 38/45; A61K 31/704; A61K 45/06; C12Y 207/10001

(57) ABSTRACT

Compositions and methods are provided for treating cancer in a mammal by administering a therapeutic dose of a pharmaceutical composition that inhibits AXL protein activity, for example by inhibition of the binding interaction between AXL and its ligand GAS6. The treatment may be combined with a cytoreductive therapy.

### CROSS REFERENCE

This application claims benefit of U.S. Provisional Patent Application No. 62/426,016, filed Nov. 23, 2016, which application is incorporated herein by reference in its entirety.

### FEDERALLY SPONSORED RESEARCH AND DEVELOPMENT

This invention was made with Government support under contract CA088480 awarded by the National Institutes of Health. The Government has certain rights in the invention.

### BACKGROUND OF THE INVENTION

Invasion and metastasis are serious and life-threatening aspects of cancer; however, primary non-invasive tumors also pose substantial and life-threatening risks. While tumors with minimal or no invasion may sometimes be successfully removed, this is not always the case. As such, therapies are needed which not only target invasive carcinoma and metastasis, but also primary tumors as well.

Therapeutic efforts in cancer prevention and treatment are being focused at the level of signaling pathways or selective modulatory proteins. Protein kinase activities, calcium homeostasis, and oncoprotein activation are driving signals and therefore may be key regulatory sites for therapeutic intervention. Kinases in signaling pathways regulating invasion and angiogenesis may be important regulators of metastasis. One of the largest classes of biochemical molecular targets is the family of receptor tyrosine kinases (RTKs). The most common receptor tyrosine kinase molecular targets to date are the EGF and vascular endothelial growth factor (VEGF) receptors. Newer kinase molecular targets include the type III RTK family of c-kit, and abl. Inhibitors of these molecules have been administered in combination with classic chemotherapeutics.

With few exceptions, curative treatment protocols in clinical oncology remain reliant upon a combination of surgical resection, ionizing radiation, and cytotoxic chemotherapy. However, in many cases the full potential of these modalities is limited by off-target effects and dose-limiting toxicities. Even when side effects can be effectively managed, durable responses are difficult to achieve, particularly in cases characterized by refractory, metastatic disease. To address these shortcomings, there has been a trend in drug discovery to develop targeted therapies capable of modulating signaling axes dysregulated in cancers. There are now many FDA approved antibodies and small molecules that allow for therapeutic manipulation of a myriad of clinically relevant targets. Collectively, these drugs have proven beneficial but not transformative; metrics of efficacy are often measured in progression-free survival rather than improved overall survival. To-date, the complex biology which drives tumorigenesis has been, for the most part, unyielding to single-agent, targeted treatments. While limited as monotherapies, the value of these drugs lies in their ability to be used with the classic aforementioned treatment modalities. By augmenting standard treatment protocols with inhibitors targeting signaling pathways known to be important within a particular patient, meaningful improvements in efficacy have been obtained within a small subset of individuals. However, most patients remain refractory even to these combination treatments, emphasizing the need for new molecular entities that have both direct anti-tumor activity, but more importantly, act synergistically with surgery, radiation, and/or chemotherapy.

One target that has shown promise in many cancers is Axl, a member of the TAM family of receptor tyrosine kinases that also includes Tyro3 and Mer. Upregulated in many forms of cancer, Axl overexpression has been linked to metastasis, poor survival, and drug resistance. Critically, Axl deficient mice have mild phenotypes, suggesting complete abrogation of signaling through the Axl receptor would confer minimal on-target toxicity. Furthermore, Axl has a single ligand, Gas6, and constitutive activation is rarely observed in tumors, leaving Gas6-mediated signaling as the primary driver of pathogenesis. Nevertheless, an unusually strong binding affinity between Gas6 and Axl of ˜30 pM has made the development of competitive antagonists challenging.

It has been shown that administration of a soluble Axl decoy receptor is an effective therapeutic strategy that circumvents the native affinity barrier. The Axl receptor contains two distinct Gas6 binding epitopes; a high affinity site on its N-terminal immunoglobulin-like (Ig) domain and a low affinity site on the second Ig domain. Previously, we engineered the major site on Axl Ig1 using a combination of rational and combinatorial protein engineering methods. The result of these efforts was MYD1, a high affinity Axl variant containing four mutations that conferred improved binding to Gas6. The characterization of MYD1 revealed a strong correlation between Gas6 binding affinity and therapeutic efficacy of the Axl decoy receptor in pre-clinical models of cancer metastasis.

Human AXL is a 2,682-bp open reading frame capable of directing the synthesis of an 894-amino acid polypeptide. Two variant mRNAs have been characterized, transcript variant 1 may be accessed at Genbank, NM_021913.3 and transcript variant 2 may be accessed at NM_001699.4. The polypeptide sequence of the native protein is provided as SEQ ID NO:1, and specific reference may be made to the sequence with respect to amino acid modifications. Important cellular functions of GAS6/AXL include cell adhesion, migration, phagocytosis, and inhibition of apoptosis. GAS6 and AXL family receptors are highly regulated in a tissue and disease specific manner.

Agents that act on this pathway for treatment of primary tumors and metastases are of great clinical and humanitarian interest.

Patent documents U.S. Pat. Nos. 8,618,254; 9,074,192; 9,266,947; 20160108378; 20160266136; 20150315552; 20150315553; 20130189254; WO2015/030849; WO2014/093690; WO2014/093707; WO2014/035828; WO2013/090776; WO2011/091305; are herein specifically incorporated by reference for all teachings.

### SUMMARY OF THE INVENTION

Compositions and methods are provided for the treatment of cancer. In the methods of the invention, administration of a cytoreductive agent or therapy, which include without limitation radiation therapy, e.g. radiation therapy; chemotherapy; and the like, including without limitation DNA damaging therapies; in combination with administration of an effective dose of an agent that is a soluble AXL variant polypeptide, wherein the AXL polypeptide lacks the AXL transmembrane domain and has at least one mutation relative to wild-type that increases affinity of the AXL polypeptide binding to GAS6 compared to wild-type AXL.

The dose of AXL variant may be sufficient to enhance the anti-proliferative effects of the cytoreductive therapy on the targeted tumor, for example where the reduction in viable tumor cells following treatment is greater than the reduction in the absence of the AXL variant. The combination of AXL variant and cytoreductive therapy may be synergistic, where the effectiveness of the combination is greater than the additive activity of the AXL variant or the cytoreductive therapy administered as a single modality.

The dose of AXL variant may be sufficient to reduce the side effects of the cytoreductive therapy on the patient, for example where there is a reduction in radiation-induced pneumonitis, hepatitis, and other radiation-specific effects following treatment, relative to the side-effects in the absence of the AXL variant.

In some embodiments, the soluble AXL polypeptide is a soluble AXL variant polypeptide, wherein said soluble AXL variant polypeptide lacks the AXL transmembrane domain, lacks a functional fibronectin (FN) domain, has one Ig1 domain, lacks a functional Ig2 domain and wherein said AXL variant polypeptide exhibits increased affinity of the AXL variant polypeptide binding to GAS6 compared to wild-type AXL.

In some embodiments, the AXL variant polypeptide is a fusion protein comprising an Fc domain. In some embodiments, the variant polypeptide lacks the AXL intracellular domain. In some embodiments, the soluble AXL variant polypeptide further lacks a functional fibronectin (FN) domain and wherein said variant polypeptide exhibits increased affinity of the polypeptide binding to GAS6. In some embodiments, the soluble AXL variant polypeptide comprises at least one amino acid modification relative to the wild-type AXL sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO:1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) T381, 8) T44A, 9) H61Y, 10) D65N, 11) A72V, 12) S74N, 13) Q78E, 14) V79M, 15) Q86R, 16) D87G, 17) D88N, 18) 190M or 190V, 19) V92A, V92G or V92D, 20) 197R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the AXL variant polypeptide comprises amino acid changes relative to the wild-type AXL sequence (SEQ ID NO: 1) at the following positions: (a) aspartic acid 87 and (b) valine 92.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the AXL variant polypeptide comprises amino acid changes relative to the wild-type AXL sequence (SEQ ID NO: 1) at the following position: alanine 72.

In some embodiments, in the AXL variant polypeptide glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, in the AXL variant polypeptide aspartic acid 87 residue is replaced with a glycine residue or valine 92 residue is replaced with an alanine residue or a combination thereof.

In some embodiments, in the AXL variant polypeptide alanine 72 residue is replaced with a valine residue.

In some embodiments, in the AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the AXL variant comprises amino acid changes relative to the wild-type AXL sequence (SEQ ID NO: 1) at the following positions: (a) glutamic acid 26; (b) valine 79; (c) valine 92; and (d) glycine 127.

In some embodiments, in the AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, in the AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, in the AXL variant polypeptide comprises amino acid changes relative to the wild-type AXL sequence (SEQ ID NO: 1) at the following positions: (a) glycine 32; (b) aspartic acid 87; (c) alanine 72; and valine 92.

In some embodiments, in the AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide has an affinity of at least about 1×10⁻⁸ M, 1×10⁻⁹ M, 1×10⁻¹⁰ M, 1×10⁻¹¹ M or 1×10⁻¹² M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide further comprises a linker. In some embodiments, the linker comprises one or more (GLY)₄SER units. In some embodiments, the linker comprises 1, 2, 3 or 5 (GLY)₄SER units.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1C. Engineering and characterization of a second generation Axl decoy receptor. (FIG. 1A) The first immunoglobulin domain of the Axl receptor was engineered for improved affinity to Gas6. When administered, the engineered soluble Axl sequesters Gas6, preventing it from binding to and activating endogenous cell surface expressed Axl. (FIG. 1B) Gas6/MYD1-72 1:1 cocomplex. Gas6 is shown in grey and MYD1-72 in blue. V72 is highlighted in red, and its location on the structure is indicated (arrows). (FIG. 1C) Cutaway showing A72 on the MYD1 and V72 on the MYD1-72. The sidechains of both are shown as dotted spheres, illustrating the space occupied by the larger valine mutation. The new interaction gained in the MYD1-72 structure is shown to the right.

FIG. 2A-2D. Superior efficacy of the second generation Axl decoy receptor. (FIG. 2A) Western blots showing the reduction of Axl and FLT3 phosphorylation in AML cells when treated with the Axl decoy receptors. (FIG. 2B) Inhibition of cell growth and induced cytotoxicity in both OCI-AML3 and MV4:11 cells after treatment with Axl Fc's. Effects are dependent upon dosage and the affinity of the decoy receptor, but not influenced by FLT3 status. Untreated data is the same in the 100 ng/mL and 10 ng/mL graphs. (FIG. 2C) In vivo sequestration of Gas6 following a single 0.5 mg/kg dose of MYD1 Fc (grey) or MYD1-72 Fc (blue). The PK profile of MYD1-72 Fc following a single 1 mg/kg dose is overlaid in red. (FIG. 2D) Amount of lung metastases in the 4T1 breast cancer model as quantified by ex vivo bioluminescent imaging. Error bars represent±stdev, n=11 for in vivo experiments, *P<0.05. Repeated measure ANOVA was used for measurement over time and student t-test was used for comparing single treatment to the control.

FIG. 3A-3G. Axl inhibition reduces primary tumor growth. (FIG. 3A) Volume over-time of orthotopically implanted primary 4T1 tumors in mice treated with vehicle, Foretinib, BGB324, or MYD1-72 Fc. (FIG. 3B) Mass of the primary tumor at the conclusion of the study. (FIG. 3C) Kaplan-Meier curve for the MYD1-72 Fc and Foretinib treated groups only. Toxicity from Foretinib treatment required half of the mice to be prematurely removed from the study. (FIG. 3D-3G) representative images and matched quantification of Ki67, TUNEL, γH2AX, and vWF staining of primary tumor tissue. Error bars represent±stdev, n=6-12, *P<0.05, **P<0.01, ***P<0.001, ****P<0.0001. Scale bars are 50 μm. Repeated measure ANOVA was used for measurement over time and ANOVA with Tukey-Kramer test was used for comparing multiple treatment to each other.

FIG. 4A-4D. Inhibition of Axl decreases metastatic tumor burden. (FIG. 4A) Lung metastases in the 4T1 model, quantified ex vivo by bioluminescent imaging. (FIG. 4B) Representative bioluminescent images of whole lungs from mice in each treatment group. (FIG. 4C) Average animal weight in each treatment group over the course of the study. Foretinib was omitted as animals were removed throughout the study. (FIG. 4D) Western blot analysis of OVCAR8 cells after 4 hour treatment with BGB324, Foretinib, or MYD1-72 Fc. Activation of all three TAM receptors as well as downstream Akt signaling was assayed. Error bars represent±SEM, n=6-12, **P<0.01. Repeated measure ANOVA was used for measurement over time and ANOVA with Tukey-Kramer test was used for comparing multiple treatment to each other.

FIG. 5A-5F. Treatment with MYD1-72 induces DNA damage response during cell S phase. (FIG. 5A) Immunofluorescence staining of γH2AX foci formation in ovarian cancer cells treated with MYD1-72 Fc alone or in combination with doxorubicin. n=7-9. (FIG. 5B) Western analysis was carried out to examine changes in the phosphorylation of ATM, ATR, CHK1, CHK2 and RPA32 in ovarian cancer cells cultured in low serum (0.1% FBS) after treatment with MYD1-72 Fc. (FIG. 5C) Representative images of EdU and γH2AX positive cells and quantification of γH2AX positive cells in MYD1-72 Fc vs. control treated cells (FIG. 5D). The differences in the number of S-phase cells (EdU positive) between control and MYD1-72 Fc treated groups are shown. No click refers to the negative control for EdU staining (FIG. 5E). The percentage of γH2AX positive cells that are in Sphase (EdU positive) between control and MYD1-72 Fc treated group are shown in (FIG. 5F). n=3. *P<0.05, **P<0.01, ***P<0.001, ****P<0.0001. Scale bars are 15 μm. ANOVA with Tukey-Kramer test was used for comparing multiple treatment to each other and student t-test was used for comparing single treatment to the control.

FIG. 6A-6C. MYD1-72 Fc enhances the effects of chemotherapy in ovarian cancer. Inhibition of metastasis in the OVCAR8 (FIG. 6A) and SKOV3.ip (FIG. 6B) ovarian cancer models as measured by number of metastases and overall tumor weight. Blue data points represent mice estimated to have over 1000 metastases, and red outlined data points represent mice with no evidence of disease. Inlaid graphs are expanded views of a subset of the complete dataset to highlight differences between treatment groups. The eyes, liver, lungs, and kidneys of mice in the OVCAR8 study (FIG. 6C) were analyzed for histological signs of toxicity. Treatment with MYD1-72 Fc did not result in ocular toxicity, as the integrity of the RPE (arrows) was maintained no histological abnormalities were present across treatment groups. Representative images are shown from each treatment group. n=7-10. *P<0.05, **P<0.01, ***P<0.001, ****P<0.0001. Scale bars are 50 μm. ANOVA with Tukey-Kramer test was used for comparing multiple treatment to each other.

FIG. 7. Inhibition of Axl in pancreatic cancer prolongs survival when used in combination with Gemcitabine. Kaplan-Meier of the LM-P orthotopic pancreatic cancer study. Animals were treated with either vehicle, MYD1-72 Fc, Gemcitabine, or MYD1-72 Fc+Gemcitabine. § P<0.0001 for combination vs. vehicle, † P<0.0001 for combination vs. Gemcitabine. A log-rank (Mante-Cox) test was performed to compare mean survival among groups.

FIG. 8A-8D. Inhibition of Axl in pancreatic cancer improves the efficacy of Gemcitabine. Representative images and matched quantification of (FIG. 8A) Ki67, (FIG. 8B) vWF, (FIG. 8C) TUNEL, and (FIG. 8D) γH2AX staining of primary tumor tissue. Error bars represent±stdev, n=10-14, *P<0.05, **P<0.01, ****P<0.0001. Scale bars are 50 μm. ANOVA with Tukey-Kramer test was used for comparing multiple treatment to each other.

### DEFINITIONS

In the description that follows, a number of terms conventionally used in the field of cell culture are utilized extensively. In order to provide a clear and consistent understanding of the specification and claims, and the scope to be given to such terms, the following definitions are provided.

The term “tumor,” as used herein, refers to all neoplastic cell growth and proliferation, whether malignant or benign, and all pre-cancerous and cancerous cells and tissues.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, the cells of interest for detection, analysis, classification, or treatment in the present application include precancerous (e.g., benign), malignant, pre-metastatic, and non-metastatic cells.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

The “pathology” of cancer includes all phenomena that compromise the well-being of the patient. This includes, without limitation, abnormal or uncontrollable cell growth, primary tumor growth and formation, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

Depending on the nature of the cancer, an appropriate patient sample is obtained. As used herein, the phrase “cancerous tissue sample” refers to any cells obtained from a cancerous tumor. In the case of solid tumors which have not metastasized (for example a primary tumor), a tissue sample from the surgically removed tumor will typically be obtained and prepared for testing by conventional techniques.

The definition of an appropriate patient sample encompasses blood and other liquid samples of biological origin, solid tissue samples such as a biopsy specimen or tissue cultures or cells derived there from and the progeny thereof. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

Tumors of interest for treatment with the methods of the invention include solid tumors, e.g. carcinomas, gliomas, melanomas, sarcomas, and the like. Breast cancer is of particular interest. Carcinomas include the a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (usually as secondary metastases), lung, breast, skin, etc. Including in the designation of soft tissue tumors are neoplasias derived from fibroblasts, myofibroblasts, histiocytes, vascular cells/endothelial cells and nerve sheath cells. Tumors of connective tissue include sarcomas; histiocytomas; fibromas; skeletal chondrosarcoma; extraskeletal myxoid chondrosarcoma; clear cell sarcoma; fibrosarcomas, etc. Hematologic cancers include leukemias and lymphomas, e.g. cutaneous T cell lymphoma, acute myeloid leukemia (AML), chronic myeloid leukemia (CML), acute lymphoblastic leukemia (ALL), non-Hodgkins lymphoma (NHL), etc.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

Anti-proliferative, or cytoreductive therapy is used therapeutically to eliminate tumor cells and other undesirable cells in a host, and includes the use of therapies such as delivery of ionizing radiation, and administration of chemotherapeutic agents. Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g. the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Nucleotide analogs include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g. cisplatin (cis-DDP), carboplatin, oxaliplatin, etc.; ureas, e.g. hydroxyurea; gemcitabine, and hydrazines, e.g. N-methylhydrazine.

For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells. Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

“Concomitant administration” of a known cancer therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

The AXL variant may be administered prior to, concurrently with, or following the cytoreductive therapy, usually within at least about 1 week, at least about 5 days, at least about 3 days, at least about 1 day. The AXL variant may be delivered in a single dose, or may be fractionated into multiple doses, e.g. delivered over a period of time, including daily, bidaily, semi-weekly, weekly, etc. The effective dose will vary with the route of administration, the specific agent, the dose of cytoreductive agent, and the like, and may be determined empirically by one of skill in the art. A useful range for i.v. administered polypeptides may be empirically determined, for example at least about 0.1 mg/kg body weight; at least about 0.5 mg/kg body weight; at least about 1 mg/kg body weight; at least about 2.5 mg/kg body weight; at least about 5 mg/kg body weight; at least about 10 mg/kg body weight; at least about 20 mg/kg body weight; or more.

As used herein, the phrase “disease-free survival,” refers to the lack of such tumor recurrence and/or invasion and the fate of a patient after diagnosis, with respect to the effects of the cancer on the life-span of the patient. The phrase “overall survival” refers to the fate of the patient after diagnosis, despite the possibility that the cause of death in a patient is not directly due to the effects of the cancer. The phrases, “likelihood of disease-free survival”, “risk of recurrence” and variants thereof, refer to the probability of tumor recurrence or spread in a patient subsequent to diagnosis of cancer, wherein the probability is determined according to the process of the invention.

The compounds having the desired pharmacological activity may be administered in a physiologically acceptable carrier to a host to modulate AXL/GAS6 function. The therapeutic agents may be administered in a variety of ways, orally, topically, parenterally e.g. intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Inhibitors,” “activators,” and “modulators” of AXL or its ligand GAS6 are used to refer to inhibitory, activating, or modulating molecules, respectively, identified using in vitro and in vivo assays for receptor or ligand binding or signaling, e.g., ligands, receptors, agonists, antagonists, and their homologs and mimetics.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g. mouse, rat, etc.

The definition of an appropriate patient sample encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, kidney disease cells, inflammatory disease cells and/or transplant rejection (GVHD) cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient. A biological sample comprising a sample cell from a patient can also include normal, non-diseased cells.

The term “diagnosis” is used herein to refer to the identification of a molecular or pathological state, disease or condition, such as the identification of a virus infection.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, covers any treatment of any virus infection or exposure in a mammal, particularly in a human, and includes: (a) preventing the infection; (b) inhibiting the infection, i.e., arresting its development; and (c) relieving the disease, i.e., causing regression of infection.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in the treatment of virus exposure or infection.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

AXL, MER, Tyro3 and GAS6, as well as related pathways, have been described in WO2011/091305, as well as U.S. application Ser. Nos. 13/554,954 and 13/595,936; all of which are incorporated herein by reference in their entireties for all purposes.

### DETAILED DESCRIPTION OF THE EMBODIMENTS

Methods of the present invention include treating, reducing, or preventing primary tumor growth or formation of primary cancer, or metastasis of cancers, by administering a soluble AXL variant polypeptide as described herein. Administration may be combined with one or more additional cytoreductive therapies. The combination may be synergistic. The combination may increase the therapeutic index of the cytoreductive therapy. The cytoreductive therapy may act in a DNA repair pathway.

Cancers of interest include solid tumors and hematologic malignancies, e.g. leukemias and lymphomas; and include without limitation primary AML, primary ovarian cancer, primary breast cancer, primary lung cancer, primary liver cancer, primary colon cancer, primary gallbladder cancer, primary pancreatic cancer, primary prostate cancer, primary ovarian cancer, and/or primary glioblastoma. Methods of the present invention also include treating, reducing, or preventing tumor metastasis.

The AXL variant polypeptide is optionally combined with an additional cytoreductive agent in pharmaceutical compositions suitable for therapeutic use, e.g., for human treatment. In some embodiments, pharmaceutical compositions of the present invention include one or more therapeutic entities of the present invention or pharmaceutically acceptable salts, esters or solvates thereof or any prodrug thereof. In some other embodiments, pharmaceutical compositions of the present invention include one or more therapeutic entities of the present invention in combination with another cytotoxic agent, e.g., another anti-tumor agent. In yet some other embodiments, pharmaceutical compositions of the present invention include one or more therapeutic entities of the present invention in combination with another pharmaceutically acceptable excipient.

In still some other embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

In yet other embodiments, methods of the present invention include administering to a subject in need of treatment a therapeutically effective amount or an effective dose of a therapeutic entity (e.g., inhibitor agent) of the present invention. In some embodiments, effective doses of the therapeutic entity of the present invention, e.g. for the treatment of primary or metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In some embodiments, the dosage may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing primary tumor formation or tumor metastasis or tumor invasion of AML, ovarian cancer, breast cancer, lung cancer, liver cancer, colon cancer, gallbladder cancer, pancreatic cancer, prostate cancer, and/or glioblastoma.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if there is a recurrence of the cancer.

According to the present invention, compositions for the treatment of primary or metastatic cancer can be administered by parenteral, topical, intravenous, intratumoral, oral, subcutaneous, intraarterial, intracranial, intraperitoneal, intranasal or intramuscular means. The most typical route of administration is intravenous or intratumoral although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. An exemplary composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein. Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration. Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent, for example a cytoreductive drug. The compositions may be provided in a unit dose formulation. Kits typically include a label indicating the intended use of the contents of the kit. The term label includes any writing, or recorded material supplied on or with the kit, or which otherwise accompanies the kit.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

### Augmenting the Efficacy of Chemotherapies by Inhibiting the Gas6/Axl Pathway

The Axl receptor and its activating ligand, growth arrest-specific 6 (Gas6), are important drivers of metastasis and therapeutic resistance in human cancers. Given the critical roles that Gas6 and Axl play in refractory disease, this signaling axis represents an attractive target for therapeutic intervention. However, the strong picomolar binding affinity between Gas6 and Axl, and the promiscuity of small molecule inhibitors, represent important challenges faced by current anti-Axl therapeutics. To overcome these obstacles, we built upon our previous work and engineered a second generation high-affinity Axl ‘decoy receptor’ with an apparent affinity to Gas6 of 93 femtomolar. Our engineered decoy receptor, MYD1-72 (comprising substitutions Gly32Ser, Ala72Val, Asp87Gly, Val92Ala, and Gly127Arg), significantly inhibited disease progression in aggressive pre-clinical models of human cancers, and induced cell killing in leukemia cells. When directly compared to the most advanced anti-Axl small molecules in the clinic, MYD1-72 achieved superior anti-tumor efficacy while displaying no toxicity. Importantly, we uncovered a novel relationship between Axl and the cellular response to DNA damage whereby abrogation of Axl signaling leads to an accumulation of the DNA damage markers γH2AX, p53BP1 and Rad51. Using our engineered molecule, this new relationship was exploited to improve the therapeutic index of current standard-of-care chemotherapies in preclinical models of advanced disease.

Here, we report the engineering and characterization of a second generation Axl decoy receptor which binds both mouse and human Gas6 significantly tighter than our original molecule. Using both engineered proteins, we further define the correlation between the affinity of the decoy to Gas6 and anti-tumor efficacy achieved in vivo. Significantly, the second generation decoy receptor was well-tolerated and outperformed the current lead clinical Axl small molecule inhibitor when directly compared. Finally, we uncovered a new relationship between Axl and the DNA damage response and leveraged this to improve the therapeutic index of standard-of-care cytotoxic chemotherapies in pre-clinical models of pancreatic and ovarian cancers.

Engineering a second-generation decoy receptor. In our original work, an error-prone library was created using the wild-type Axl Ig1 domain as a template, and placed into the yeast display system. After six rounds of flow cytometric sorting, the library was enriched for three Axl variants with improved binding to Gas6. While only three variants were present after the sixth and final round of sorting, significant diversity was retained in earlier sort products. To evaluate whether further increased Gas6 binding affinity could improve anti-tumor efficacy, we performed sequence analysis on the sort five products to identify additional gain-of function mutations. A total of 141 clones were sequenced, yielding 25 unique variants. Three mutations were found to occur in at least 20% of these unique variants: A72V, D87G, and V92A, of which the latter two are contained in MYD1. When mapped onto the Gas6/MYD1 structure (PDB: 4RA0), residue 72 is distant from the four mutations already contained in MYD1. Furthermore, the structural rearrangements observed on the Gas6/MYD1 structure do not occur near position 72. The high frequency of A72V in the enriched pool indicated that it likely improved binding to Gas6, and its isolation in 3-dimensional space on the structure suggested it would improve the affinity of MYD1 to Gas6.

To test this empirically, we recombinantly expressed an Axl Ig1 variant containing the A72V point mutation. The A72V mutation did not disrupt the overall folding of the protein (Figure S1) and the mutant was determined to bind Gas6 with an affinity of 5.8 pM, five times stronger than wild-type Axl Ig1 (Table 1). Encouraged by these results, A72V was then combined with MYD1 yielding an Axl variant (MYD1-72) with an affinity for Gas6 of 720 fM (Table 1). Using thermodynamic cycle analysis, contributions from A72V were determined to be nearly completely additive with the preexisting mutations in MYD1, illustrating the independent nature of this mutation. When reformatted as an Fc fusion, MYD1-72 Fc had an apparent binding affinity to human Gas6 of 93 fM, a 350-fold increase over wild-type Axl, and also bound more strongly to mouse Gas6 with an apparent affinity of 140 fM (Table 1).

Structural basis of high-affinity binding. To elucidate the structural origins underlying the affinity increase, the Gas6/MYD1-72 cocomplex was crystallized. This structure, along with the wild-type (PDB: 2C5D) and MYD1 (PDB: 4RA0) co-complexes, use the Axl Ig1-Ig2 and Gas6 LG1-LG2 fragments to produce a 2:2 co-complex. Despite significant effort and successful growth of crystals under several different conditions, only a low resolution structure (3.5 Å) was obtained. While the low resolution precluded detailed analysis of inter-residue contacts, no significant backbone changes were observed relative to the MYD1 structure (Cα r.m.s.d. 0.2 Å), particularly within the regions around residue 72 and within Helix A on Gas6.

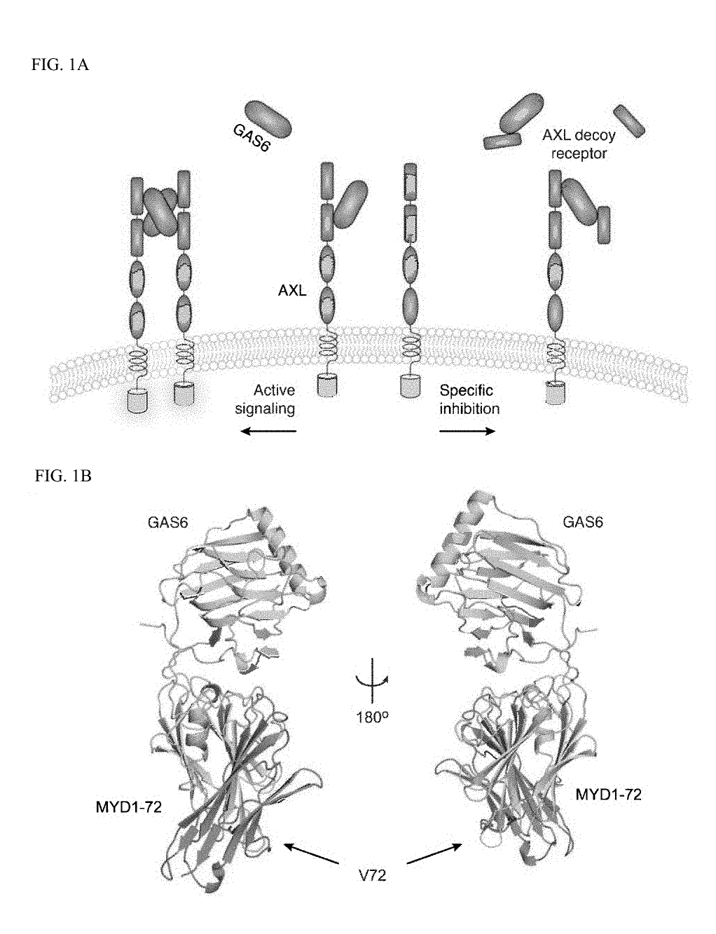

In an effort to improve crystal quality, we simplified the structure by truncating Axl, complexing only MYD1-72's high-affinity Ig1 domain with Gas6, and were able to generate a high-resolution structure at 2.3 Å (FIG. 1B). When compared to the Gas6/MYD1 structure (PDB: 4RA0, at 3.4 Å), the intermolecular contacts across the interface were similar. Importantly, analysis of the binding interface at high resolution supports previous conclusions using the Gas6/MYD1 structure. Specifically, the N-terminal capping of Helix A on Gas6 that we reported in the MYD1 co-complex was conserved in the MYD1-72 co-complex. These observations serve as structural confirmation that A72V and the preexisting mutations in MYD1 act independently.

While the intermolecular contacts at the binding interface were similar between the two structures, significant differences in the region around position 72 were observed. First, in the MYD1-72 structure a single additional electrostatic interaction was observed on the backside of the complex within the loop containing position 72 (FIG. 1C). Second, local packing within the core of Axl is markedly different (FIG. 1C), as the increased volume of the valine side chain permits more efficient side chain packing and thus a lower void volume. As a result, several van der Waals contacts are gained, further strengthening the core structure of MYD1-72. Together, these two features likely serve as the structural basis for the improvements in affinity seen in the A72V mutant.

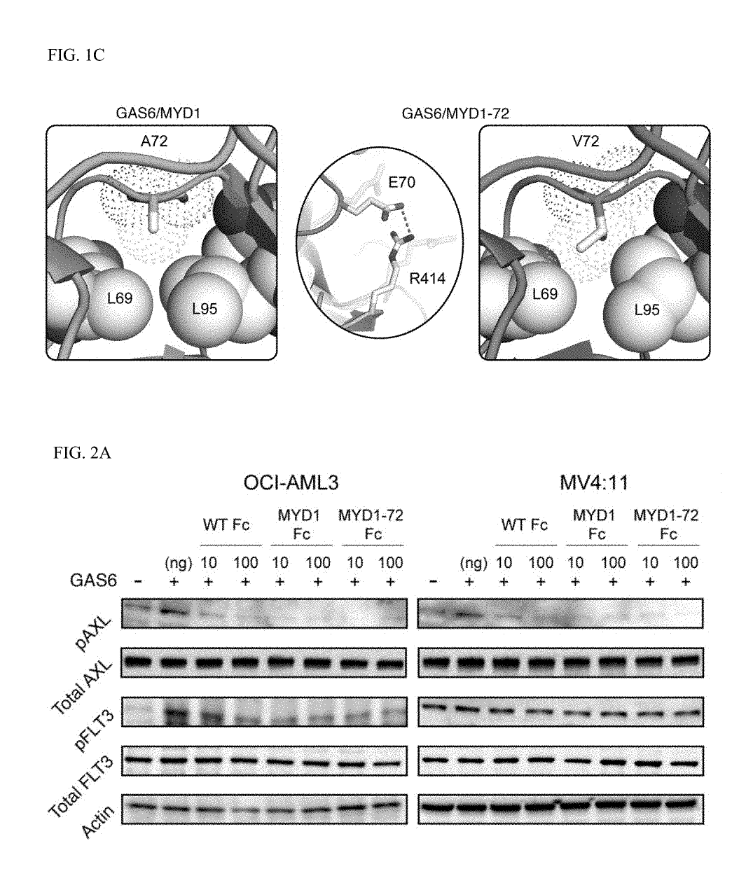

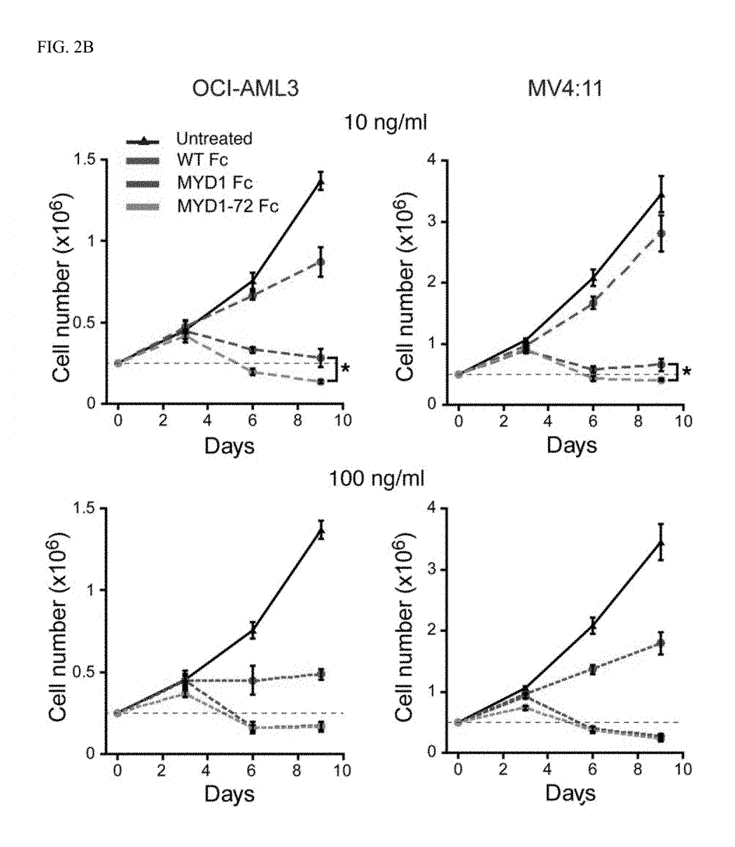

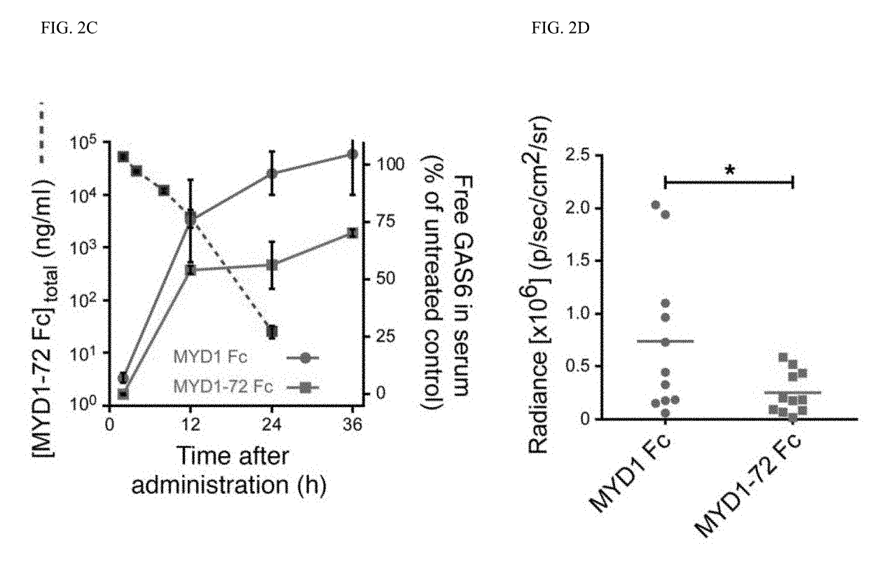

MYD1-72 Fc has improved anti-tumor efficacy. Axl is a known survival factor in acute myeloid leukemia (AML), as cytotoxicity is seen when signaling through the receptor is antagonized. To test whether the enhanced affinity of MYD1-72 Fc would equate to increased activity, we initially characterized the effects of the decoy receptors on the in vitro growth of two human AML cell lines: OCI-AML3 and MV4:11. While OCI-AML3 cells are wild-type for FLT3, MV4:11 cells contain an internal tandem duplication (ITD) of the FLT3 receptor, resulting in constitutive activation. FLT3 ITD remains a significant clinical challenge and is associated with poor prognosis. Treatment with wildtype Axl Fc, MYD1 Fc, or MYD1-72 Fc inhibited Axl and FLT3 phosphorylation (FIG. 2A), and cytotoxicity was observed in a dose dependent manner, independent of FLT3 status (FIG. 2B). Importantly, the improved affinity of MYD1-72 Fc compared to MYD1 Fc correlated with enhanced activity (FIG. 2B).

To determine if the increase in activity would translate in vivo, we evaluated how well MYD1 Fc and MYD1-72 Fc could systemically sequester endogenous Gas6. To ensure that potential improvements could be quantified, a dose of 0.5 mg/kg was used, which is the in vivo IC₅₀ of MYD1 Fc as previously determined using the same assay. Mice were administered MYD1 Fc or MYD1-72 Fc and serum samples were obtained at time points up to 36 hours post injection. The amount of free, circulating Gas6 (i.e. not neutralized by the decoy receptor) was then quantified. Both molecules rapidly eliminated free Gas6 upon administration, though MYD1-72 Fc suppressed Gas6 levels longer than MYD1 Fc (FIG. 2C), highlighting its improved pharmacodynamic (PD) profile. These improvements were not due to differences in the clearance rates of the molecules, as the PK profile MYD1-72 Fc was similar to what was previously reported for MYD1 Fc (FIG. 2C).

To directly study the consequences of MYD1-72 Fc's improved PD, we used both decoy receptors as treatments in the 4T1-luciferase breast cancer model of metastasis. In this model, cells orthotopically implanted in the mammary fat pad generate primary masses that metastasize to the lungs. Four days after tumor implantation, mice were treated with either MYD1 Fc or MYD1-72 Fc at the sub-optimal dose of 0.5 mg/kg. After three weeks of treatment, mice receiving MYD1-72 Fc had significantly less metastatic disease than those treated with MYD1 Fc (FIG. 2D). Collectively, these studies demonstrate the superior efficacy of our second generation molecule, further emphasizing the important correlation of the decoy receptor's affinity to therapeutic efficacy.

Comparison of MYD1-72 Fc with clinical Axl. TKIs There are currently no FDA approved drugs targeting Axl, though several small molecule kinase inhibitors are undergoing clinical trials. BerGenBio's BGB324 (previously Rigel's R428) is the most advanced of these compounds, and the first to be developed prospectively as an Axl inhibitor. Given the positive clinical results seen thus far with BGB324, we hypothesized that it would serve as a good benchmark of comparison for MYD1-72 Fc. Furthermore, since TKI's and biologics are fundamentally very different, as are their mechanisms of action, a direct comparison would provide valuable insights into the potential of distinct therapeutic intervention strategies. We therefore compared the anti-tumor effects of MYD1-72 Fc to BGB324 and another TKI with significant activity against Axl, Foretinib, in the 4T1-luciferase model. Cells were implanted orthotopically in the mammary fat pad, and primary tumors were allowed to establish prior to treatment. Mice were then randomized into one of four treatment groups: saline, MYD1-72 Fc daily at 1 mg/kg, BGB324 twice daily at 12.5 mg/kg, or Foretinib twice daily at 12.5 mg/kg. This dose for the TKIs was chosen as significant activity has been previously reported at equivalent concentrations.

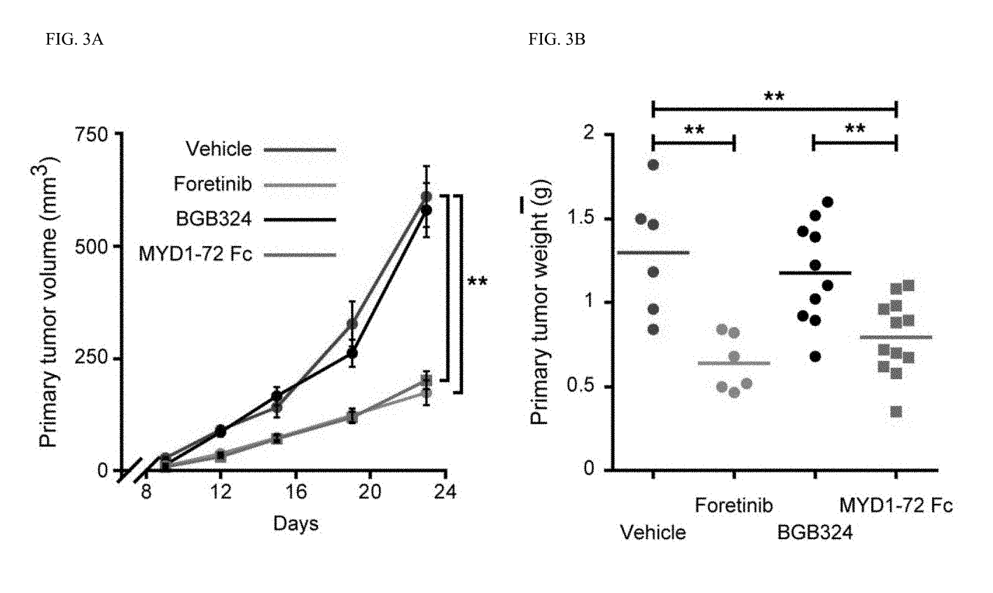

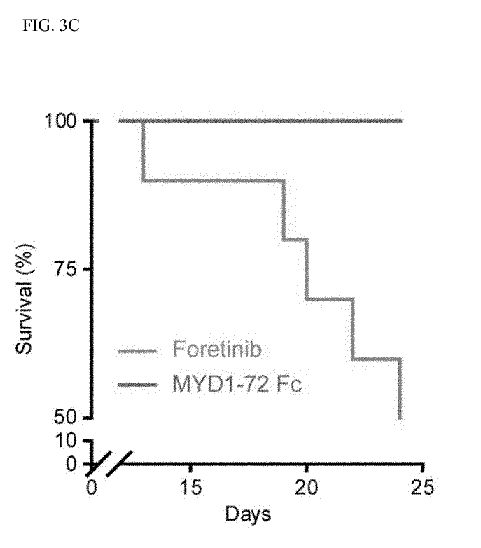

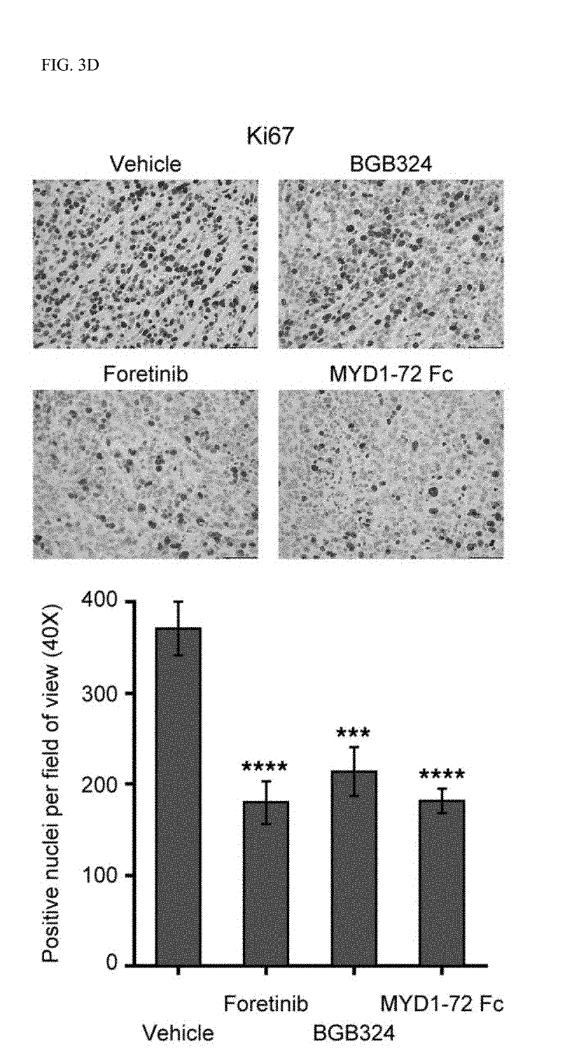

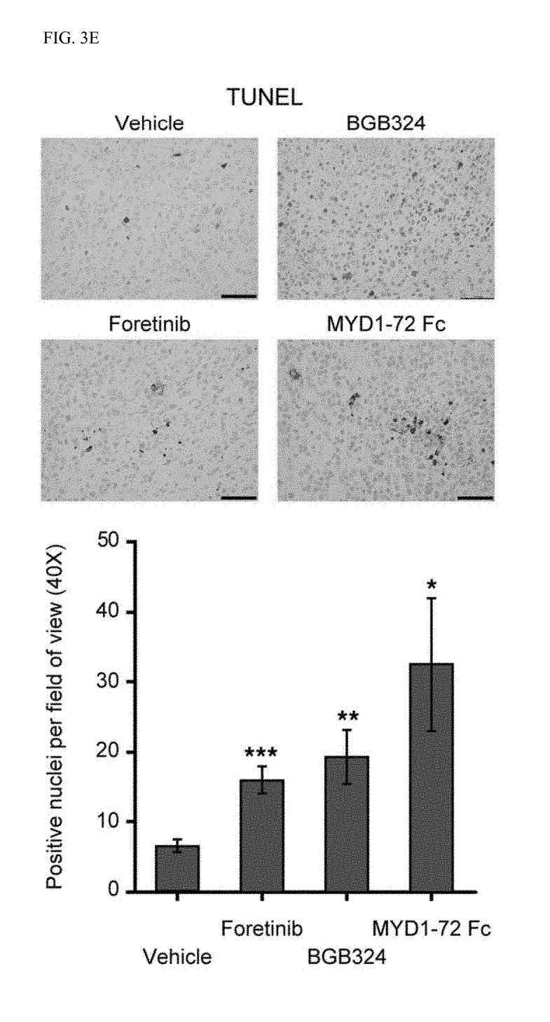

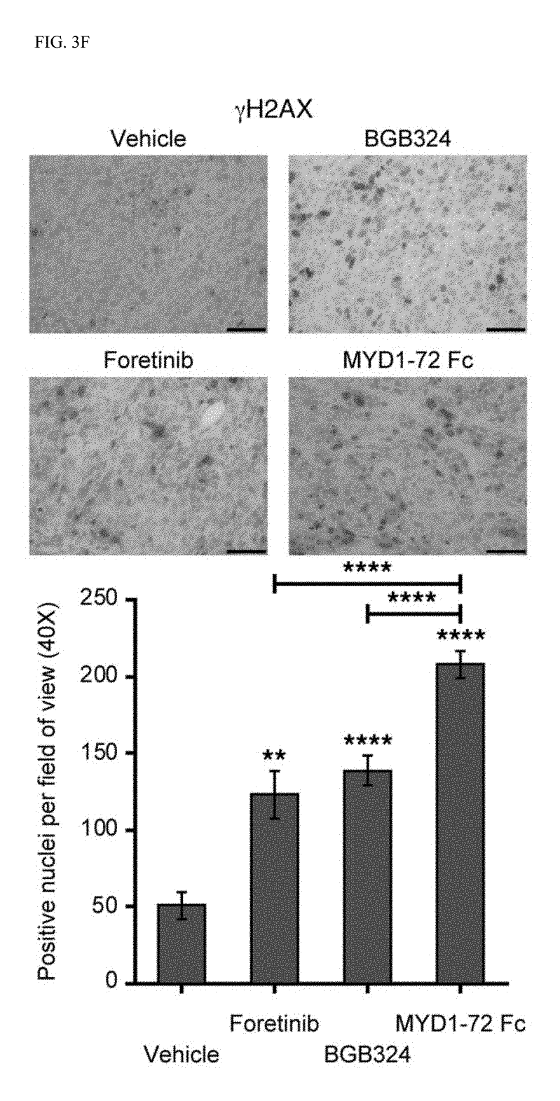

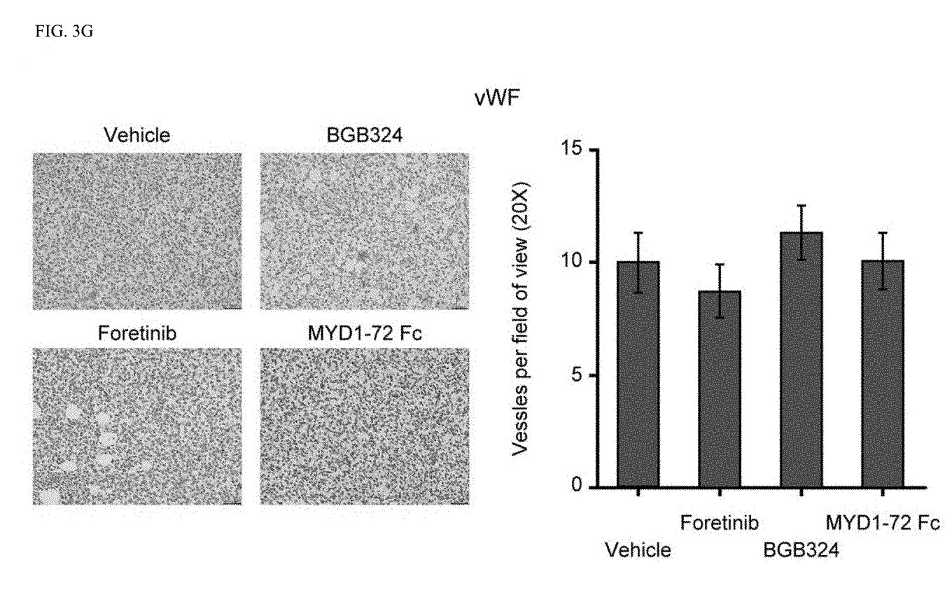

Mice were treated for three weeks, or until they showed significant signs of morbidity at which point they were removed from the study. Both MYD1-72 Fc and Foretinib significantly reduced the size of primary tumors compared to control mice, while BGB324 showed little effects (FIGS. 3, A and B). This was notable because with the exception of AML, Axl has historically been a driving force of metastatic disease, showing little effect on cellular growth in vitro or primary tumor growth in vivo. Though anti-tumor efficacy was seen in the Foretinib treated group, substantial toxicity was also present, requiring half of the mice to be sacrificed prior to the study endpoint (FIG. 3C). Immunohistochemistry was performed on sections of primary tumor from each treatment group to study the mechanism underlying the reduction in growth. Staining for the proliferation marker Ki67 revealed all treatment groups had significantly less proliferation within the primary tumor, compared to control animals (FIG. 3D). Complimenting this, there was also more intratumoral apoptosis in the treatment groups (FIG. 3E). To further understand the increased apoptosis rate, we stained γH2AX, a marker of DNA double-strand breaks. All treatment groups showed elevated levels of γH2AX compared to controls; however tissue samples from MYD1-72 Fc treated animals had significantly more γH2AX positive cells compared to all other treatment groups (FIG. 3F). While Axl has been linked to VEGF signaling and endothelial cells, no changes in vessel density were observed across the groups (FIG. 3G).

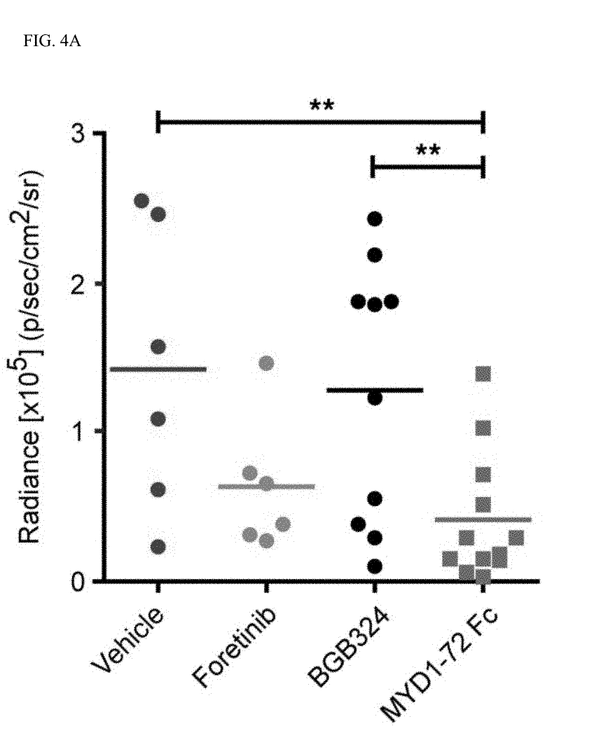

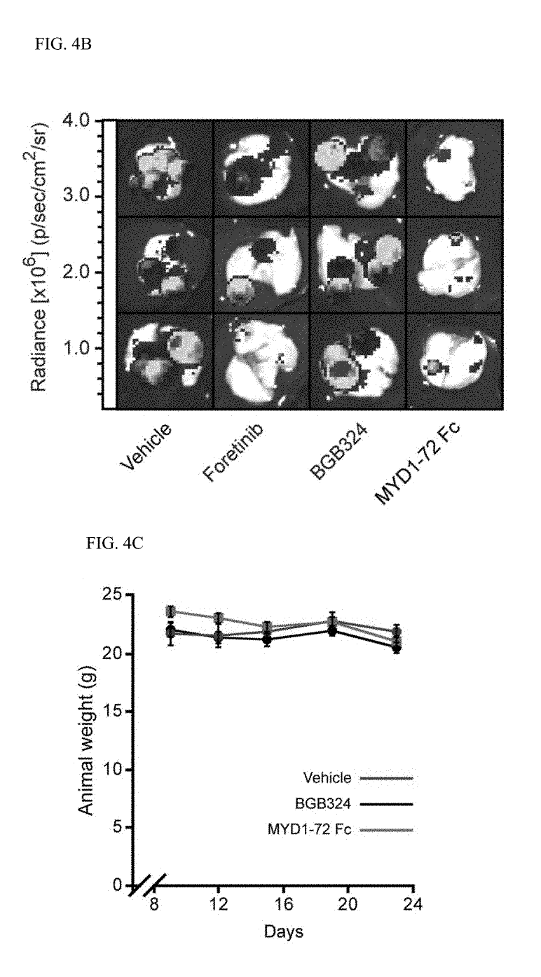

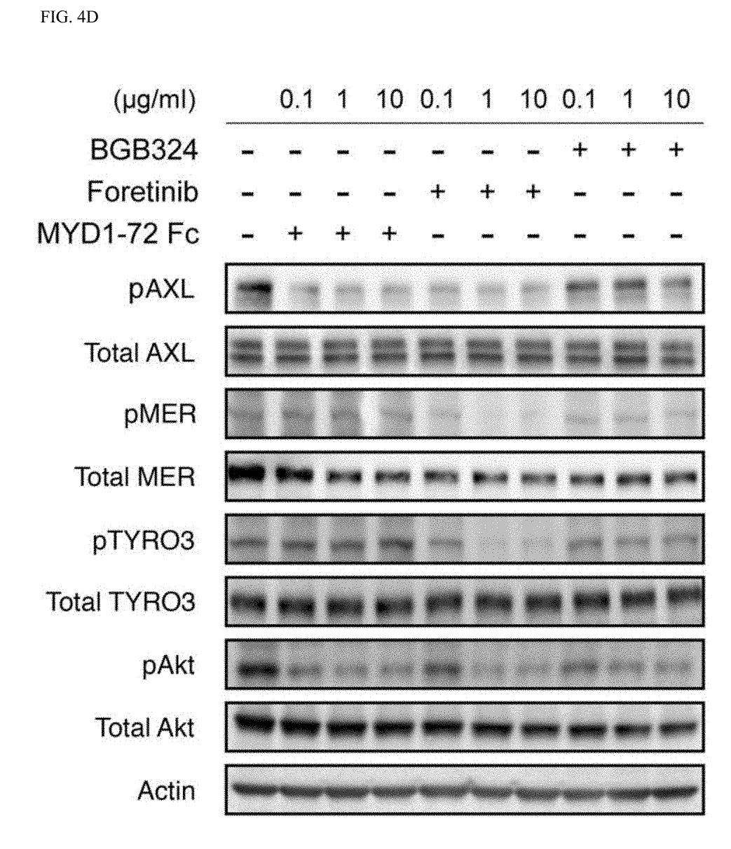

MYD1-72 Fc and Foretinib also demonstrated activity against metastatic disease, as seen by a 71% and 55% reduction in lung metastases, respectively, compared to vehicle treated mice (FIGS. 4, A and B). In contrast, BGB324 had a modest 10% decrease that was not significantly different than control animals. Throughout the study, both MYD1-72 Fc and BGB324 were well tolerated with no visible signs of toxicity, as animal weight remained consistent across treatment groups (FIG. 4C).

To further understand these differences between MYD1-72 Fc, Foretinib, and BGB324, we examined changes in the phosphorylation of Axl, Mer, Tyro3, and downstream Akt signaling in vitro upon treatment with each. MYD1-72 Fc significantly reduced pAxl and pAkt levels, while pMer and pTyro3 remained relatively unchanged (FIG. 4D). These data indicate that MYD1-72 Fc is specific at attenuating the Axl signaling cascade, achieving significant efficacy without noticeable off-target effects. In contrast, BGB324 treatment resulted in short-term inhibition of pAxl but increased pAxl levels at later time points (FIG. 4D). While efficacious, the promiscuity of Foretinib, which indiscriminately inhibited all TAM receptors (FIG. 4D), was likely responsible for the significant toxicity observed. Collectively, these data demonstrate that MYD1-72 Fc compares favorably to anti-Axl TKIs currently being evaluated in the clinic, both in terms of safety and efficacy. Specifically, MYD1-72 Fc recapitulates the efficacy of Foretinib without the aforementioned toxicity, while achieving greater anti-tumor effects than BGB324.

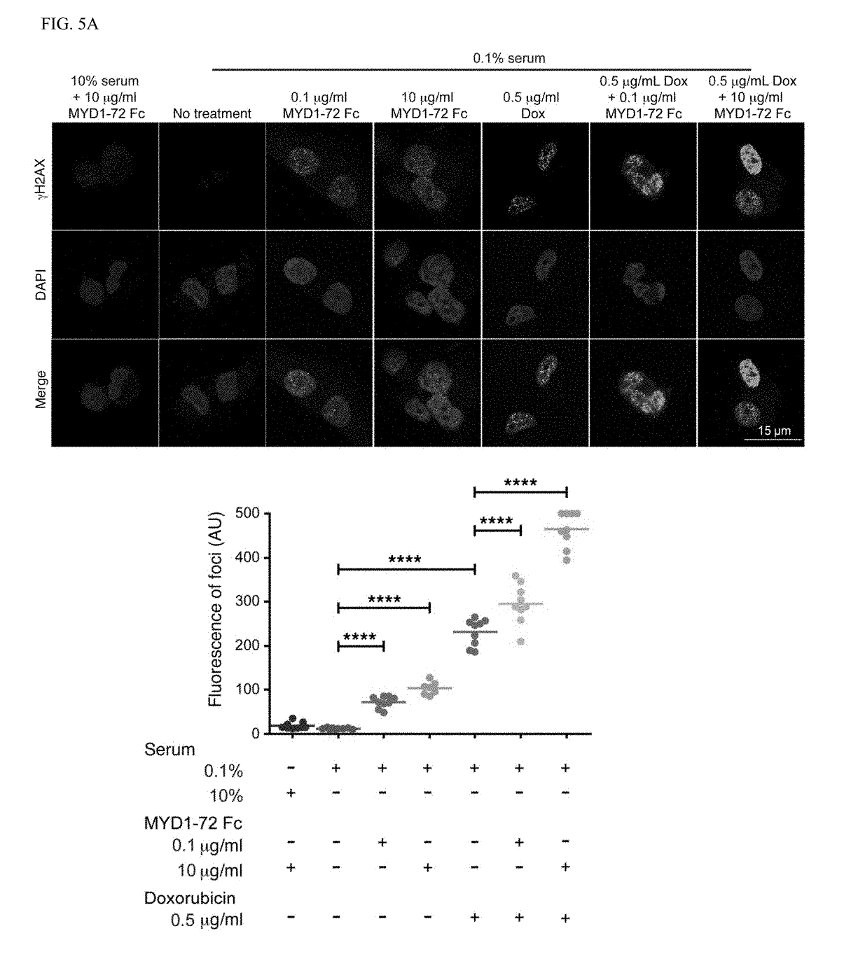

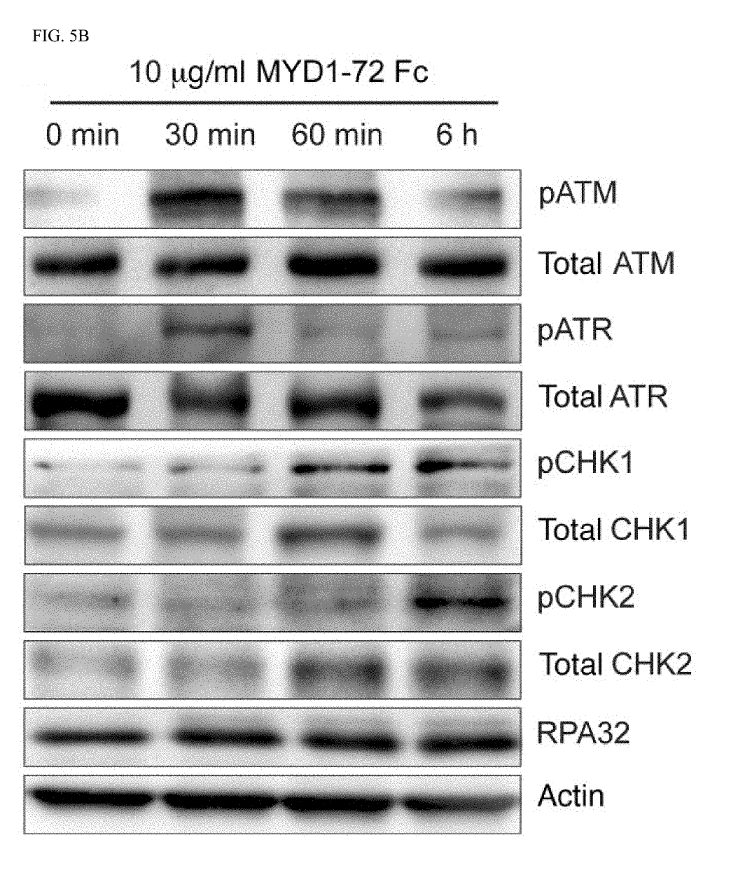

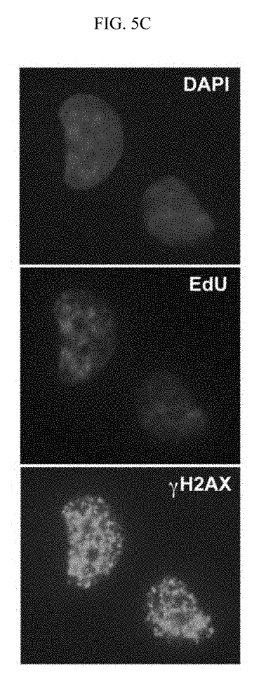

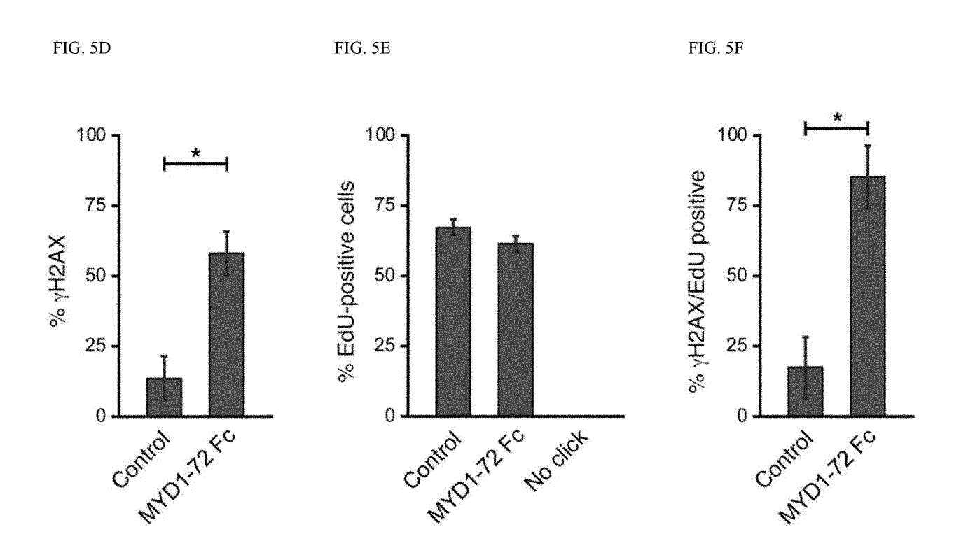

Axl signaling augments the DNA damage response. Axl signaling is known to be a critical driver of tumor progression and drug resistance, leading to interest in combining Axl inhibitors with other targeted therapies. Our data suggests that inhibiting Axl induces a DNA damage response, as demonstrated by elevated γH2AX levels in the primary tumors of the 4T1 study (FIG. 3F). To further interrogate the link between Axl and the DNA damage response, we first performed immunofluorescence staining to detect γH2AX foci in ovarian cancer cells treated with 0.1 μg or 10 μg of MYD1-72 Fc. While cells treated with MYD1-72 Fc in complete media showed no changes across treatments (FIG. 5A), treatment done under serum-limited conditions resulted in a significant increase in the number of γH2AX foci compared to untreated controls (FIG. 5A), suggesting that a DNA damage response is activated upon inhibition of Axl signaling.

To further understand this relationship, we performed a reverse phase protein array (RPPA) on shAxl cell lines and wild-type cell lines treated with MYD1-72 Fc. We found Akt, mTor, and P70SK6 to be differentially expressed in all datasets, demonstrating the fidelity of both assay and samples as these downstream effectors are known to be controlled in part by Axl signaling. Interestingly, key components of the DNA damage response (XRCC1/Chk2) as well as some pro-apoptotic members of the Bcl-2 pathway (BAX/BID) were highly upregulated in the shAxl cell line, and when wild-type cells were treated with MYD1-72 Fc continuously for seven days in low serum (1% FBS). In addition, we performed western blot analysis to examine expression levels of classical DNA damage response components, including total and phosphorylated ATM, ATR, CHK1, CHK2 and RPA32 (FIG. 5B) at four time points post MYD1-72 treatment. We saw significant induction of phospho-ATM and phospho-ATR at early time points, followed by increased phosphorylation of both CHK1 and CHK2. Elevated phospho-RPA32 level was also observed (as shown by the doublet band on the total RPA32 blot), suggesting replication stress. These data suggest that the loss of Axl signaling can modulate DNA damage response signaling in tumor cells under stress conditions such as nutrient deprivation.

To interrogate the nature of the DNA damage response, we immunofluorescently stained cells treated with MYD1-72 Fc for 53BP1 and RAD51 foci. Under serum-limited conditions, treatment with MYD1-72 Fc increased the numbers of both 53BP1 and RAD51 foci (Figure S5), further supporting our hypothesis that loss of Axl signaling promotes a DNA damage response. Intrigued by the RPA phosphorylation observed following Axl inhibition (FIG. 5B), we performed EdU labeling and co-staining of cells for γH2AX (FIG. 5C). These experiments would allow a better understanding of whether the γH2AX signaling previously observed following Axl inhibition was occurring in EdU positive/S-phase cells, suggesting a link between Axl inhibition and replication stress. FIG. 5D show the percentage of γH2AX positive cells upon treatment with MYD1-72 Fc. This result supports the previous observation that treatment with MYD1-72 Fc induces γH2AX signal in cancer cells. There was a minor difference in the number of S-phase cells between control and MYD-72 Fc treated groups that is unlikely to alone account for the difference in distribution of γH2AX signal (FIG. 5E). Interestingly, we observed that in the MYD-72 Fc treated group, 85% of cells that were γH2AX positive were also in S phase/EdU positive (FIG. 5F) while only 7% of non-S phase cells (EdU negative) were positive for γH2AX.

Furthermore, cells treated with hydroxyurea in the presence and absence of MYD1-72 Fc showed comparable numbers of γH2AX positive cells and no additive effect was observed when the two compounds were used in combination. Together these results show that Axl inhibition can contribute to replication stress resulting in increasing DNA damage signaling. That these effects were observed only under serum-limited conditions is further indication that Gas6/Axl signaling provides a cyto-protective effect for tumor cells placed under stress, such as growth factor deprivation. These results provide the rationale for combining Axl inhibitors and DNA damaging agents, such as radiation and chemotherapy to enhance their therapeutic index.

Inhibiting Axl improves the standard-of-care. Clinical management of ovarian cancer remains a significant challenge as patients often present with advanced metastatic disease at the time of diagnosis. Treatment for these patients is limited and entails surgical debulking followed by combination chemotherapy; however, tumor response rates remain poor. Coincidentally, ovarian cancer represents an ideal setting to test the combination of Axl inhibitors and DNA damaging agents, as patients are in critical need of new treatment options that can enhance the effects of standard-of-care chemotherapy. We therefore treated ovarian cancer cells with MYD1-72 Fc either alone or in combination with doxorubicin in vitro and assessed levels of DNA damage by staining the cells for γH2AX foci. Cells treated with MYD1-72 Fc alone showed a significant increase in the number of γH2AX foci compared to untreated controls; however, γH2AX levels were further elevated when doxorubicin was also present (FIG. 5A). These data further support the link between inhibition of Axl signaling and the DNA damage response, and suggest that a synergistic effect can be achieved when anti-Axl therapies are combined with cytotoxic chemotherapy.

To see whether these results would translate in vivo, we evaluated the efficacy of MYD1-72 Fc alone and in combination with doxorubicin in two models of human ovarian cancer, the OVCAR8 and skov3.ip models. For both models, cells were injected intraperitoneally where they were allowed to establish for one week prior to treatment. At this time, several mice were sacrificed at random to ensure engraftment of diffuse metastatic disease, a hallmark of the human condition. Mice were then randomized into one of four treatment groups: saline, MYD1-72 Fc at 1 mg/kg daily, doxorubicin at 2 mg/kg twice weekly, or the combination of MYD1-72 Fc and doxorubicin. After three weeks of treatment, mice were sacrificed and tumor burden was quantified both by counting the number of visible lesions, as well as excising and weighing all diseased tissue.

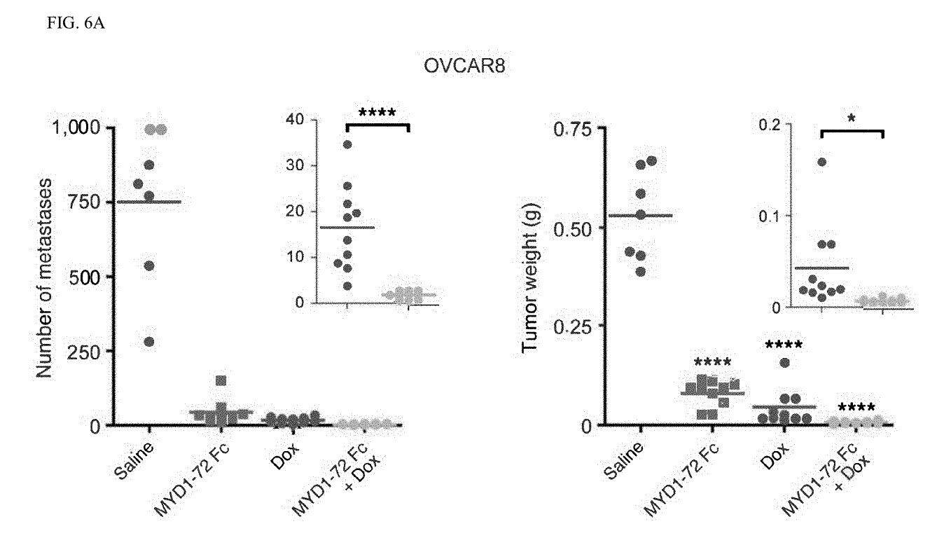

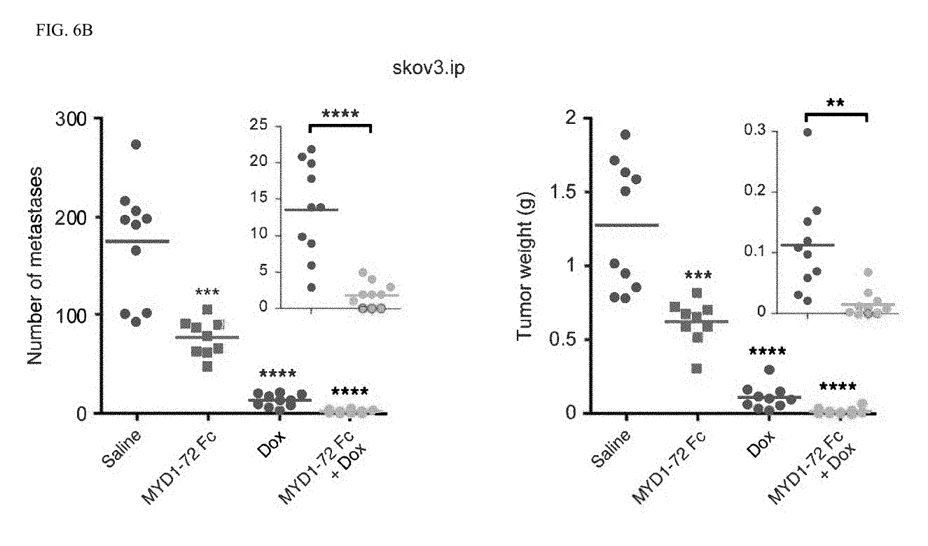

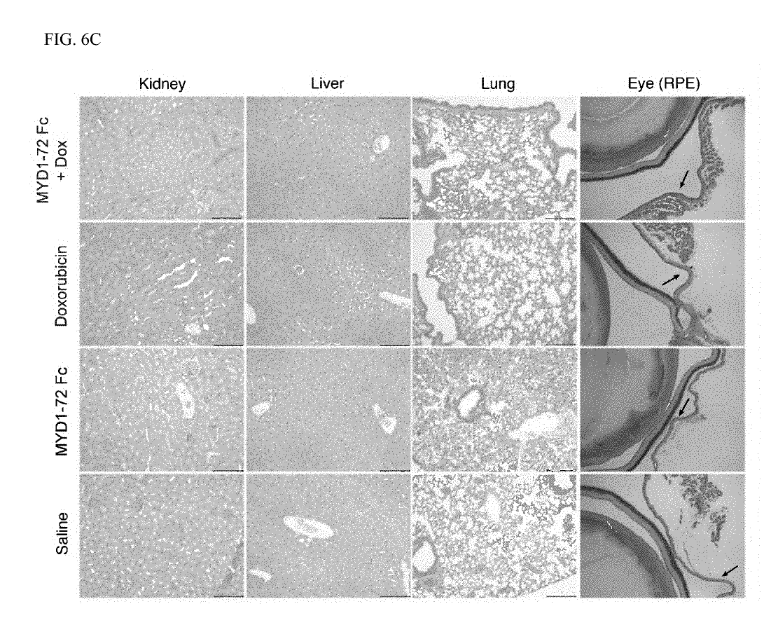

In the OVCAR8 model, MYD1-72 Fc had significant anti-tumor effects, reducing tumor burden by 95% as a single-agent (FIG. 6A). While similar effects were obtained using doxorubicin alone, combination treatment resulted in nearly undetectable levels of disease. Each mouse receiving both therapies had on average two and at most three macroscopic metastases, while the mean number in the control group was in excess of 750 (FIG. 6A, inlaid graph). A similar degree of diminishment was observed when comparing cumulative metastatic mass (FIG. 6A).

Comparatively, the skov3.ip model was more aggressive, with mice in the control group having nearly three times as much tumor tissue, by weight, at the conclusion of the study than in the OVCAR8 study (FIG. 6B). Under these conditions, using the number of nodules as a measure of tumor burden became misleading as lesions grew into one another, resulting in a smaller number of larger metastases. Even in this more advanced setting, MYD1-72 Fc and doxorubicin had significant anti-tumor activity, decreasing tumor burden by 51% and 91%, respectively (FIG. 6B). The combination of these two agents was once again effective as animals in the combination group had, on average, 99% less tumor by weight than controls. Furthermore, within this group three out of ten animals were completely cured with no evidence of disease (FIG. 6B, inlaid graphs). Together, these two studies support the in vitro findings that Axl inhibition modulates the DNA damage response, and demonstrates that antagonizing the Gas6/Axl signaling axis can be leveraged to improve the therapeutic index of chemotherapy.

Successful inhibition of Axl carries with it little risk of on-target toxicity, as seen in the Axl knockout mouse. However, broad inhibition of the TAM family can lead to severe toxicities. For example, autoimmune diseases are common in the TAM triple knockout mouse and disruption of the retinal pigmented epithelium (RPE) can lead to blindness when signaling through Mer is abrogated. Furthermore, concurrent inhibition of Axl and Mer has been shown to enhance tumor growth in certain types of cancer. To ensure the decoy receptors were specifically targeting Axl in vivo, we performed histology to assess the integrity of the RPE in mice from the skov3.ip experiment. Across all treatment groups, the RPE was healthy and normal indicating that requisite signaling through other TAM family members, specifically Mer, is preserved (FIG. 6C). Additionally, histological analysis on the liver, lung, and kidney from these animals showed no histological abnormalities across treatment groups, indicating a lack of gross toxicity (FIG. 6C).

Combination treatment improves overall survival of pancreatic cancer. Similar to recurrent ovarian cancer, pancreatic cancer represents a largely intractable clinical challenge. The primary tumor location makes early detection improbable and patients often present with advanced disease, as illustrated by five year survival rates of around 6%. Even when surgery is feasible, adjuvant chemotherapies are nearly ubiquitously administered in the form of the pyrimidine antagonists Gemcitabine or Fluorouracil (5-FU). Pancreatic cancer thus represents an additional clinical setting in which improved therapies are desperately needed and standard treatment includes DNA damaging agents. To examine whether the synergistic effects seen in the ovarian models were indicative of a general phenomenon broadly applicable to clinical oncology, MYD1-72 Fc was tested alone and in combination with Gemcitabine in an orthotopic model of murine pancreatic cancer. LMP cells derived from the KRAS/p53 metastatic mouse model were implanted subcutaneously into the flanks of mice and allowed to grow until they reached ˜500 mm³ in size.

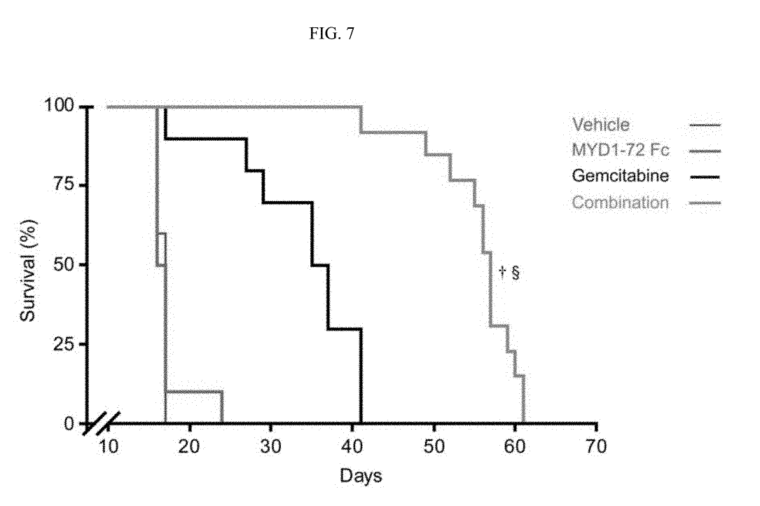

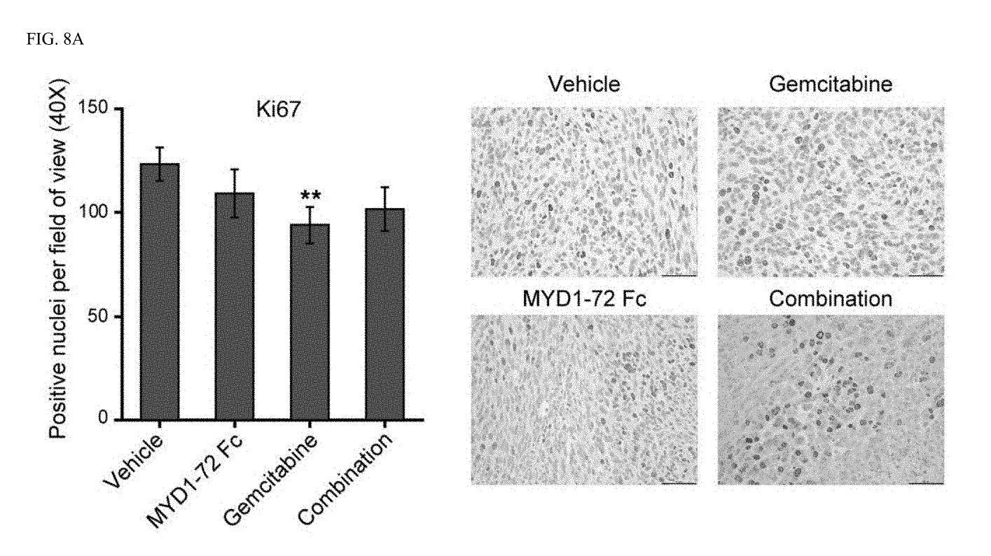

To establish orthotopic tumors, mice harboring subcutaneous tumors were sacrificed and tumors were isolated and cut into small fragments. Laparotomies were then performed and a tumor fragment was secured to the tail of the pancreas. Four days after engraftment, mice were randomized into one of four treatment groups: saline, MYD1-72 Fc at 1 mg/kg daily, Gemcitabine at 100 mg/kg twice weekly, or a combination of MYD1-72 Fc and Gemcitabine. Dosing continued until a mouse showed significant signs of morbidity at which time it was removed from the study. Direct engraftment of tumor tissue to the pancreas yielded a rapidly progressing primary tumor, with a median survival in the control group of seventeen days (FIG. 7, and Table 2). As single agents, MYD1-72 Fc showed no activity with a median survival of seventeen days, while Gemcitabine doubled median survival to thirty-five days. As in the ovarian models, combining MYD1-72 Fc and chemotherapy showed significantly greater efficacy over either therapy alone, as median survival was tripled to fifty-seven days (FIG. 7, and Table 2). Across treatment groups, animals succumbed to large primary tumor masses, rather than diffuse metastatic disease.

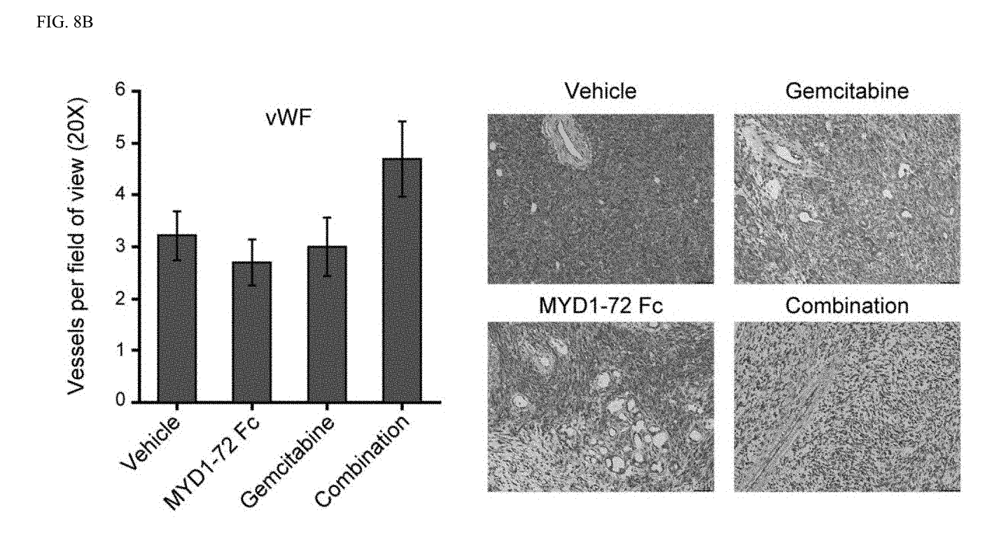

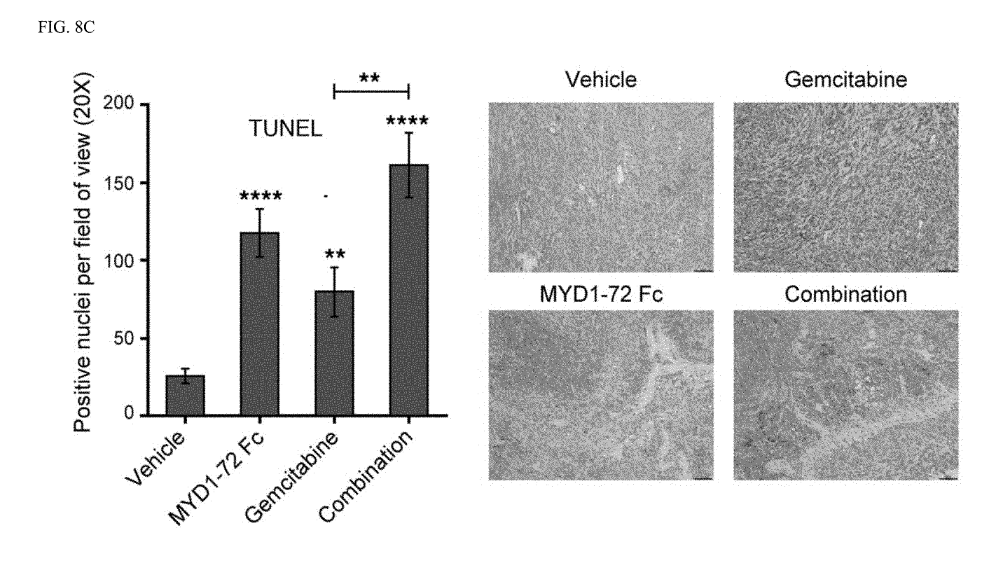

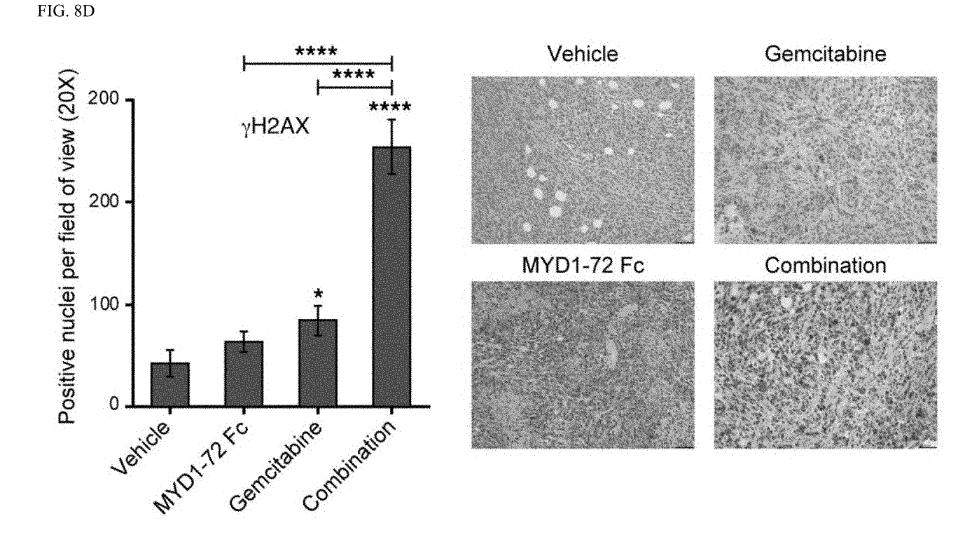

To determine if increased sensitivity to DNA damage was the mechanism driving the effects seen in the combination group, immunohistochemistry was performed on primary tumor tissue samples. Ki67 staining showed a small, albeit significant decrease in proliferation within the Gemcitabine treated group (FIG. 8A), while vessel density remained unchanged (FIG. 8B). Intratumoral apoptosis was significantly increased in all treatment groups compared to controls, particularly in those animals administered MYD1-72 Fc despite the fact that the decoy receptor alone demonstrated negligible effects on overall survival (FIG. 8C). Most notably, although MYD1-72 Fc treatment had a small effect on DNA damage, the combination of MYD1-72 Fc and Gemcitabine significantly increased the amount of γH2AX staining compared to all other groups (FIG. 8D). These results strengthen the link between Axl inhibition and the DNA damage response, and in combination with the data from the 4T1 and ovarian cancer models, highlight it as an important mechanism across cancer types. Furthermore, along with the ovarian cancer studies, these data convincingly demonstrate that this relationship can be exploited to achieve meaningful improvements in overall response rates over what can be realized by current clinical standards-of-care.

TABLE 1

| | Gas6 binding parameters | | | |
|---|---|---|---|---|
| | K_{d} (fM) | | | K_{d} (fM) |
| | hGas6 | kₒₙ | k_{off} | mGas6 |
| | *apparent | (10⁷ M⁻¹ s⁻¹) | (10⁻⁵ s⁻¹) | apparent |
| wt Axl Ig1^{§} | 33,000 | 2.1 | 70 | n.d |
| MYD1 Ig1^{§} | 2,700 | 1.6 | 4.0 | n.d |
| A72V Ig1 | 5,800 | 1.9 | 11.0 | n.d |
| MYD1-72 Ig1 | 720 | 1.7 | 1.2 | n.d |
| MYD1-Fc^{§} | *420 | 2.3 | 1.0 | 1,100 |
| MYD1-72Fc | *93 | 2.7 | 0.25 | 140 |

TABLE 2

| | median survival |
|---|---|
| treatment | (days) |
| vehicle | 17 |
| MYD1-72 Fc | 17 |
| Gemcitabine | 35 |
| combination | 57 |

As our understanding of the molecular basis of cancer has improved, a number of dysregulated signaling pathways responsible for driving disease progression have been identified. Efforts have been made to exploit these pathways as targets for therapeutic intervention, with the expectation that drugs capable of modulating them would deliver previously unachievable efficacy. In practice, however, the genetic instability and hypermutation rates of cancer, coupled with the redundancy often built in to biological systems, have undermined the importance of singular targets. In the presence of this confluence of factors, resistant mutations arise and compensatory signaling pathways become upregulated, limiting the utility of specific inhibitors. As an illustration, clinical trials for some new molecular entities prospectively set progression-free survival as the primary endpoint, highlighting the difficulty in achieving meaningful advancements in overall survival.

These observations suggest that while the development of new targets is critical, priority should be given to those which have the potential to act in synergy with, and increase the therapeutic index of, established treatment modalities. The Axl receptor fits this description. Axl initially attracted attention because of the fundamental roles it plays in driving tumor progression and metastatic dissemination, but more recently relationships between the receptor and other disease pathways have emerged. The intersection of these two features has thus made Axl an ideal oncology target.

There is a critical need for the development and characterization of anti-Axl therapeutics. To this end, a pipeline of Axl antagonists has been generated that is comprised largely of small molecule kinase inhibitors. The paucity of biologics under clinical development can be attributed to the comparatively poor binding affinity of the monoclonal antibodies (mAbs) described to-date, as the strength of the native Gas6/Axl interaction is orders of magnitude stronger than each mAb. As an example, several generations of anti-Axl antibodies were developed with affinities to the receptor in the nanomolar range. When tested in preclinical models, modest anti-tumor effects were seen, hinting at the value of the target while highlighting the difficulties inherent to inhibiting Axl.

We have previously demonstrated that the use of an engineered Axl decoy receptor is an effective way to exploit the native interaction itself in order to overcome this affinity barrier. Here, by developing a second-generation decoy receptor we further validate this approach and more clearly define the relationship between the affinity of our Axl antagonists and anti-tumor effects achieved in vivo. This re-engineered molecule, MYD1-72, combines an additional beneficial mutation, allowing it to attain a sub-picomolar affinity to Gas6. Comparisons of the first and second generation receptors, demonstrated that this improved binding affinity was critical for achieving optimal efficacy. Importantly, at 93 fM, the apparent affinity of the final MYD1-72 Fc construct represents one of the strongest protein-protein interactions reported.

An additional feature of MYD1-72 is that it binds strongly to both mouse and human Gas6. The original MYD1 showed no increase in binding to mouse Gas6, preventing it from effectively neutralizing endogenous mGas6 in pre-clinical models. By binding both orthologs with exceptionally high affinity, MYD1-72 overcomes this challenge, and thus the efficacy obtained in our mouse models is more representative of what could be achieved clinically.

One major challenge when interpreting the results of preclinical models is that their multivariable nature precludes an accurate assessment of efficacy in the absence of a known, internal control. Therefore, to more appropriately determine its clinical potential, we placed MYD1-72 head-to-head with leading anti-Axl TKIs. We demonstrate that in vitro, MYD1-72 Fc specifically inhibits Axl signaling without affecting Mer or Tyro3 activities, whereas the TKIs were more promiscuous. Foretinib and BGB324 are both known as Axl inhibitors, though their ability to attenuate Axl signaling differs dramatically. At the same dosage, Foretinib was indiscriminant, inhibiting all three TAM receptor family members, whereas BGB324 treatment resulted only in modest inhibition of Axl signaling. In all cases, downstream phospho-Akt expression was suppressed, suggesting that BGB324 can elicit its therapeutic efficacy by influencing alternative signaling cascades. These different activity profiles are likely due to two critical factors: MYD1-72 Fc only binds to Gas6, leaving ProteinS and other ligands present in the serum free to bind to Mer and Tyro3, and the polypharmacology of the anti-Axl TKIs result in broader kinase inhibition.

One critical detail of this study was the dosages that were chosen. Initial studies outlining the development of BGB324 achieved efficacy at 7 mg/kg twice daily, but more consistently used dosages in excess of 100 mg/kg twice daily. This high dose is in line with what is often used when evaluating TKIs pre-clinically, most likely because a negative result at these elevated doses would be definitive. However, clinically the total daily dose of approved TKIs in oncology is, with few exceptions, less than 12 mg/kg. Though differences in how a drug behaves in mice and humans subjects invariably exist, doses that exceed what is realistically achievable in the clinic by over an order of magnitude can skew pre-clinical results. As a result, we chose to dose both Foretinib and BGB324 at 12.5 mg/kg twice daily. For MYD1-72 Fc, we dosed mice at 1 mg/kg daily. We have previously shown that this dosing regimen is equivalent to 10 mg/kg administered bi-weekly, and thus chose daily dosing as it reduced the total protein administered by nearly two-thirds.

When directly compared, the efficacy of MYD1-72 Fc was equivalent to that of the best TKI tested, Foretinib. However, severe toxicity undermined Foretinib's results as half of the treatment group was removed from the study prior to the predetermined endpoint due to excessive morbidity. This highlights one of the confounding issues of using elevated doses of TKIs; the high homology of kinases results in significant off-target activity of even the most specific inhibitors. This promiscuity was observed in the clinic as well. In a recent phase II trial, Foretinib failed to demonstrate efficacy despite the observation of treatment-related adverse events in 91% of patients. Furthermore, within our study, BGB324 was largely ineffective when dosed at 12.5 mg/kg twice a day, having only a marginal effect on metastatic disease. It should be reiterated that this dose of BGB324 is on the low end of what has been reported to be effective. The studies on clinical dose escalation have not been published and it is possible that higher doses of BGB324 would be effective, should they prove to be tolerable.

Collectively, these data demonstrate that the specificity of our biologic allows significant anti-tumor activity to be achieved with little toxicity. These effects are equivalent to what can be attained by TKIs at their maximally tolerated dose, but carry few, if any, of the off-target risks of those compounds.

An unexpected result from the 4T1 study was that treatment with MYD1-72 Fc substantially reduced primary tumor growth. Immunohistochemical analysis of primary tumor samples showed elevated levels of γH2AX, leading us to investigate the link between Axl and the DNA damage response. Using a reverse phase protein array, a new relationship between Axl signaling and the DNA damage response was uncovered, wherein inhibition of Axl resulted in increased levels of DNA damage markers such as Chk2, XRCC1, BAX, and BID.

Furthermore, we demonstrated that the loss of Axl signaling upon treatment with MYD1-72 Fc leads to phosphorylation of the classical DNA damage response components ATM, ATR and RPA32. By performing an EdU incorporation assay with hydroxyurea and MYD1-72 Fc, we uncovered a new role for Axl signaling in protecting cancer cells from disruptive replication and fork collapse during S phase. Based on these data, we concluded that the Gas6/Axl signaling cascade provides cyto-protection for tumor cells, and loss of Axl removes this protection leading to replication stress and subsequent activation of the DNA damage response. We showed that these effects were significantly more pronounced when Axl was inhibited on cells cultured under serum limited conditions, as cells grown in complete growth media showed a negligible increase in γH2AX expression upon treatment with MYD1-72 Fc. This observation is insightful as it indicates that Axl's protective role is only activated under significant stress.

This provides context to the fact that upregulation of Axl is generally associated with later stage metastatic disease, situations where tumor cells are under severe nutrient and oxygen deprivation. DNA damaging agents have long been used to exploit cancer's hyperproliferative state, and modulation of the damage response through the abrogation of Axl signaling was shown to increase the therapeutic effects of these drugs both in vitro and in vivo. In fact, this effect was so pronounced that the reduction of tumor burden in models of human ovarian cancer exceeded 99%, and in some cases resulted in complete cures.

That these effects were observed in multiple tumor models suggests that the relationship between Axl and the DNA damage response is one that transcends cancer subtypes. These results offer the possibility of improving the therapeutic index of many standard-of-care chemotherapies, potentially providing a meaningful advancement in the care of many patients with refractory disease.

Moving forward, additional studies exploring whether other DNA damaging modalities, such as radiation, would benefit from concurrent Axl inhibition are justified. Furthermore, clinical studies combining candidate Axl inhibitors with cytotoxic agents would provide a clear advantage when evaluating efficacy. Collectively, this study expands the role that Axl plays in tumor progression and the development of therapeutic resistance. By implicating the receptor as an important regulator of the DNA damage response, anti-Axl therapies can be broadly used to chemo-sensitize in a myriad of malignancies. Our second generation Axl decoy receptor is poised to capitalize upon this potential by providing a molecule capable of effectively antagonizing the Gas6/Axl system, with little-to-no toxicity. Clinical evaluation of MYD1-72 Fc is therefore justified, particularly when used as an adjuvant to standard-of-care chemotherapy.

Materials and Methods:

Recombinant Protein Production

Axl Ig1 variants were produced in the methylotrophic yeast P. pastoris as previously described. Briefly, Ig1 variants were cloned into the pPIC9K plasmid (# V175-20 Thermo Fisher Scientific, Walthan, Mass.) with flanking N- and C-terminal FLAG and 6×HIS tags, respectively, and yeast were transformed according the manufacturer's protocol. Recombinant proteins were purified from conditioned culture supernatant by nickel affinity chromatography followed by removal of N-linked glycans using endoglycosidase H (EndoHf, P0703S, New England Biolabs, Ipswich, Mass.). Final purification was performed using size exclusion chromatography. Human Gas6 LG1-2 and both Axl constructs used for crystallography studies were expressed and purified as previously described. All Axl Fc fusion constructs were expressed transiently using the FreeStyle Max (Invitrogen, Carlsbad, Calif.) HEK293 system as previously described. Dimeric Fc fusions were isolated from conditioned culture supernatant using protein A affinity chromatography followed by size exclusion chromatography.

The Kinetic Exclusion Assay (KinExA)

Equilibrium binding and association rate constants for all Gas6-Axl interactions were measured on a KinExA3200 instrument (Sapidyne Instruments Inc. Boise, Id.) as previously described. For equilibrium binding studies, three independent titrations were completed for each Axl Ig1 or Axl Fc fusion unless otherwise noted. Depending on the affinity of interaction, three of the following four titrations were performed: 1) 1 ml reactions of 5 nM Gas6, Axl serially diluted 1:2 twelve times starting at 30 nM, 3 h RT incubation; 2) 2 ml reactions of 500 pM Gas6, Axl serially diluted 1:2.5 twelve times starting at 10 nM, 18 h RT incubation; 3) 12 ml reactions of 50 pM Gas6, Axl serially diluted 1:3 twelve times starting at 9 nM, 1 day RT incubation; 4) 12 ml reactions of 15 pM Gas6, Axl serially diluted 1:3 twelve times starting at 1 nM, 4 day RT incubation. After the appropriate incubation time, reactions were flowed over MYD1 Fc coated beads and captured free Gas6 was probed using an anti-6×HIS Dylight 649 antibody (#200-343-382, Rockland Immunochemicals Inc., Pottstown, Pa.). Each sample was measured twice and data from the three independent equilibrium binding experiments were globally analyzed using n-curve analysis in the KinExA Pro 3.6.2 software (Sapidyne Instruments Inc. Boise, Id.) to obtain the Kd value. To analyze the association rate of the interactions, the direct inject method was used. For these experiments, 1 μM Axl was serially diluted 1:2.5, and equal volumes of each Axl sample and 500 nM Gas6 were briefly mixed and flowed over the capture beads. Free Gas6 was detected as described and the data was fit using the KinExA Pro 3.6.2 software to obtain the association rate (kon) of the interaction. The dissociation rate (koff) was calculated as the product of the Kd and the kon.

Circular Dichroism Spectroscopy

The circular dichroism spectra of Axl Ig1 variants were measured on a Jasco J-815 circular dichroism spectropolarimeter. Recombinant proteins were diluted to 10 μM in PBS and ellipticity was measured in the far UV range from 190-260 nm at 20° C. in a quartz cuvette with a 1 mm path length. Raw data was transformed to mean residue ellipticity using the following equation:

$${[ θ ]}_{mrw} = \frac{mrw * {θ}_{obs}}{10 * 𝓁 * c}$$

where: mrw is the mean molecular weight per residue, θobs is observed ellipticity in degrees, l is the path length in centimeters, and c is the concentration of protein in grams/mL. Three independent data sets were collected and averaged to obtain the spectrum for each protein, with each data set representing the average of triplicate scans.

Crystallization and Data Collection of Gas6/Axl Co-Complexes

Crystallization for the 2:2 Gas6/MYD1-72 Ig1-2 co-complex was performed as previously described. Briefly, purified wild-type Gas6 and MYD1-72 Ig1-2 were mixed in a 1:1 molar ratio and allowed to complex at room temperature for 24 h. The co-complexes were purified using size exclusion chromatography to remove any unreacted components, and were buffer exchanged into 25 mM Na-HEPES, 150 mM NaCl, and 1 mM calcium acetate to a final concentration of 10 mg/ml. Crystals for the Gas6/MYD1-72 co-complex were grown at room temperature by the hanging-drop vapor-diffusion method with a 1:1 mixture (1.2 μl each) of the complex solution (5.6 mg/ml) and the well solution containing 0.15 M Li2SO4, 0.1 M Tris-HCl (pH 8.5), 5% glycerol, and 2 mM Ni2SO4. For cryocooling, the crystals were dipped in a solution containing 38 parts of 1M Li2SO4, 0.1 M Tris-HCl (pH 8.5), 0.1 M NaCl and 12 parts of 100% glycerol. Diffraction data sets for the Gas6/MYD1-72 co-complex (3.5 Å) were collected at 100K using Stanford Synchrotron Radiation Lightsource (SSRL) beamline 12-2 at a wavelength of 0.98 Å. Data were indexed and integrated using the XDS package. The crystals belong to space group P3221 and contain two monomers per symmetric unit. Several attempts to improve the resolution by using various additives, crystal dehydration experiments, and changes in the cryocooling procedure were unsuccessful. Nearly identical conditions were used to crystalize the 1:1 Gas6/MYD1-72 Ig1 co-complex. Briefly, purified wild-type Gas6 and MYD1-72 Ig1 were mixed in a 1:1 molar ratio and allowed to complex at room temperature for 24 h. The co-complexes were purified using size exclusion chromatography to remove any uncomplexed components, buffer exchanged into 25 mM Na-HEPES, 150 mM NaCl, and 1 mM calcium acetate to a final concentration of 10 mg/ml. Crystals for the Gas6/MYD1-72 Ig1 co-complex was grown at room temperature by the hanging-drop vapor-diffusion method with a 1:1 mixture (1.2 μl each) of the complex solution (9.8 mg/ml) and the well solution containing 0.7 M Li2SO4 and 0.1 M Tris-HCl (pH 8.4). For cryocooling, the crystals were dipped in a solution containing 38 parts of 1.1M Li2SO4, 0.1 M Tris-HCl (pH 8.4), 0.1 M NaCl and 12 parts of 100% glycerol. Before cryocooling, crystals were slightly dehydrated by placing the coverslip over 1 M Li₂SO₄, 0.1 M Tris-HCl (pH 8.4) well solution for 8 hrs. Diffraction data set for the Gas6/MYD1-72 Ig1 co-complex (2.3 Å) was collected at 100K using Stanford Synchrotron Radiation Lightsource (SSRL) beamline 12-2 at a wavelength of 0.98 Å. Data were indexed and integrated using XDS package. The crystals belong to space group P212121 and contain 2 monomers per symmetric unit. Crystals were also grown from three other crystallization conditions, but they showed weaker diffraction.

Structure Determination and Refinement

For the Gas6/MYD1-72 Ig1-2 co-complex, initial phases were obtained by molecular replacement by using the program MOLREP and the coordinates of the wild-type Gas6/Axl crystal structure (PDB ID: 2C5D) as the search model. Several cycles of manual model building using COOT and refinement using REFMAC resulted in final R (working) and Rfree values of 20.3% and 24.5%, respectively. The Ramachandran statistics are as follows: 93.28 (favored) and 1.15 (outlier). The structure of the Gas6/MYD1-72 Ig1 co-complex was similarly solved by molecular replacement, again using the coordinates of the wild-type structure as the search model. No electron density was observed for the loop of Gas6 from residues 542 to 550 in both chain A and chain B for the Gas6/MYD1-72 Ig1 co-complex; the wild type Gas6/Axl Ig1-2 crystal (PDB ID: 2C5D) structure also does not show electron density for this region. Several cycles of manual model building using COOT and refinement using REFMAC resulted in final R (working) and Rfree values of 19.8% and 24.7%, respectively. The Ramachandran statistics are as follows: 96.6% (favored) and 0.64% (outlier). All crystal structure figures were created using PyMOL.

Analysis of Intermolecular Contacts

The intermolecular contacts between Gas6 and MYD1 A72V in the co-complex were examined as described. Briefly, the binding interface was analyzed using the PDBePISA server v1.47 utilizing a cutoff of 3.5 Å and 4.0 Å for hydrogen bonds and electrostatic interactions, respectively. Analysis was performed in a manner identical to the prior Gas6/MYD1 structure to enable direct comparison.

Analysis of Van Der Waals Contacts

The van der Waals contacts between the side chain of the residue at position 72 and surrounding residues were determined using the WHAT IF server. A contact is defined as two atoms for which the distance between the van der Waals surfaces is less than 1.0 Å. The WHAT IF algorithm uses the following van der Waals radii: C: 1.8 Å; O: 1.4 Å; N: 1.7 Å; S: 2.0 Å.

In Vivo Tumor Models

All procedures involving animals and their care and use were approved by the Institutional Animal Care and Usage Committee of Stanford University. For all in vivo studies, six week old, female nude (nu/nu) mice (Jackson Laboratory, Bar Harbor, Me.) were used. In all studies, animals displaying signs of morbidity were removed from the study immediately. To establish orthotopic mammary tumors, 5×10⁴ 4 T1 luciferase cells (a kind gift from Dr. Marta Vilalta, Stanford University, Stanford, Calif.) were injected in a volume of 50 μl of DMEM into the mammary fat pad. Tumor engraftment was confirmed four days post-tumor inoculation by bioluminescence imaging using an IVIS 200 (PerkinElmer, Inc., Walthan, Mass.). Treatment was initiated four days post-inoculation for both studies. In the initial study comparing MYD1 Fc and MYD1-72 Fc, proteins were administered intravenously twice a week via tail vein injection in a volume of 200 μl at 0.5 mg/kg. In the second study comparing MYD1-72 Fc and the small molecule TKIs, proteins were administered daily via intraperitoneal injections at 1 mg/kg. BGB324 (Selleckchem LLC, Houston, Tex.) and Foretinib (MedChem, Monmouth Junction, N.J.). Small molecules were administered twice daily via oral gavage. On day 24, mice were sacrificed 10 minutes after receiving a single intraperitoneal (IP) injection of D-luciferin. Ex-vivo bioluminescent imaging of the lungs and spleen was performed using an IVIS 200 and images were analyzed in the Living Image software v4.3.1 to quantify lung metastases. In both the skov3ip (a gift from Dr. Gordon Mills at MD Anderson Cancer Center, Houston, Tex.) and OVCAR8 ovarian cancer models (NCI-Frederick DCTD tumor cell line repository), diffuse metastatic disease was established by injecting 1×106 cells intraperitoneally. Tumors were allowed to establish for one week prior to treatment, and on day five a subset of animals were sacrificed to confirm the presence of visible macroscopic disease. In both studies, MYD1-72 Fc was administered at 1 mg/kg IP daily, doxorubicin (APP pharmaceutical, INC. Schaumburg, Ill.) at 2 mg/kg was administered IP twice a week, or both were administered as described for a total of three weeks. Animals were sacrificed on day 28 and metastatic burden was assessed by counting the number of visible metastatic lesions in the peritoneal cavity as well as excising and weighting all tumor tissue. The orthotropic LM-P pancreatic adenocarcinoma model (a gift from Dr. Edgar Engleman at Stanford University, Stanford, Calif.) was established as previously described. Briefly, 1×10⁶ cells were injected subcutaneously into the flanks of nude mice and grown for 2-3 weeks until they reached 500 mm³. To establish orthotropic tumors, mice harboring the subcutaneous tumors were sacrificed and tumors were isolated and cut into small 3-4 mm fragments. Laparotomies were performed and a tumor fragment was secured to the tail of the pancreas using sutures. After implantation, the pancreas was returned to the peritoneal cavity and the incision was closed. Mice received daily injections of antibiotic on the day of implantation and on each of the three days post-op for pain management. Treatment was initiated four days post-surgery. MYD1-72 Fc was administered at 1 mg/kg IP daily, Gemcitabine (Sun Pharma, Mumbai, India) at 2 mg/kg was administered IP twice a week, or both were administered as described. Treatment was continued until a mouse displayed significant signs of morbidity at which time it was removed from the study.

In Vivo Gas6 Serum ELISA

For the time course studies, mice were administered a single dose of MYD1 Fc or MYD1-72 Fc at 0.5 mg/kg via tail vein injection. All doses were formulated in a 200 μl volume. Two mice were analyzed per condition and untreated mice were used to determine baseline Gas6 levels. At 2, 12, 24, and 36 h post-administration, retro-orbital bleeds were performed to obtain blood samples from which serum was isolated and free Gas6 was measured as previously described. The amount of free Gas6 in each sample was determined using a sandwich ELISA. In this assay, MYD1 Fc was used as a capture reagent in order to ensure the detection of free, unbound Gas6, and not Gas6/Axl Fc complexes. Detection of Gas6 was carried out using a biotinylated polyclonal anti-mouse Gas6 antibody (BAF986, R&D Systems, INC., Minneapolis, Minn.) and streptavidin HRP (#4800-30-06 Trevigen Inc., Gaithersburg, Md.).

Reverse Phase Protein Array (RPPA)

The RPPA was performed by MD Anderson as described.

Immunoblotting

Cell lysates subjected to sodium dodecyl sulfate polyacrylamide gel electrophoresis followed by transfer to nitrocellulose membrane. The membranes were then probed with primary antibodies against total Axl (AF154, R&D Systems, INC., Minneapolis, Minn.), phospho-Axl (T702; #5724, CST, Danvers, Mass.), total Akt (#4691 CST, Danvers, Mass.), phosphor-Akt (#4060, CST, Danvers, Mass.), anti-MERTK (#ab52968, Abcam, Cambridge, Mass.), phospho-MERTK (#ab14921 Abcam, Cambridge, Mass.), anti-Tyro3 (#5585, CST, Danvers, Mass.), phospho-Tyro3 (#orb186274, Biorbyt LLC, Berkely, Calif.), Bcl-2 Family Antibody Sampler Kit (#9934, CST, Danvers, Mass.), anti-Chk2 (#sc-5278, Santa Cruz Biotech, INC., Santa Cruz, Calif.), anti-XRCC1 (#2735, CST, Danvers, Mass.), anti-γH2AX (Merck Millipore #05636, Darmstadt, Germany), anti-phospho-ATM (#ab81292, Abcam, Cambridge, Mass.), anti-ATM (#ab78, Abcam, Cambridge, Mass.), anti phospho-ATR (#2853, CST, Danvers, Mass.), anti-ATR (#2790, CST, Danvers, Mass.), anti-Phospho Chk1 (#12302, CST, Danvers, Mass.), anti-Phospho Chk2 (#2197, CST, Danvers, Mass.) and anti-RPA32 (#2208, CST, Danvers, Mass.) at 4° C. overnight. The blots were then washed and probed with HRP conjugated anti-goat (#sc-2020, Santa Cruz Biotech, INC., Santa Cruz, Calif.), or HRP conjugated anti-rabbit (#A16110, Thermo Fisher Scientific, Walthan, Mass.) as appropriate. The blots were developed with Bio-Rad Western C developing reagent (#170-5060 Bio-Rad, Hercules, Calif.) and visualized with Chemidoc digital imager (#1708280, Bio-Rad, Hercules, Calif.).

Immunofluorescence Analysis

10,000 cells were plated in each well of glass chamber slide and allowed to attach overnight. Cells were serum starved and treated with 0.1 □g or 10□g of MYD1-72 Fc+/− (2.5 mg) Doxorubicin. Cells were then washed with PBS and fixed with 4% paraformaldehyde, lysed for 10 minutes and blocked with a 2% BSA and 0.1% PBS-Triton-X solution for 1 hour. After washes in PBS-Triton-X, cells were incubated with mouse anti-□H2AX, (#05-636, Merck Millipore, Darmstadt, Germany); rabbit anti-RAD51, (#PC130-100 UL Merck Millipore, Darmstadt, Germany); rabbit anti-53BP1, (# ab36823, Abcam, Cambridge, Mass.) overnight in a humidified chamber at 4 degrees. Cells were washed with PBS and incubated in secondary antibody anti-rabbit FITC (#65-6111 Thermo Fisher Scientific, Walthan, Mass. or anti-mouse FITC, (#62-6511, Thermo Fisher Scientific, Walthan, Mass.) for 1 hour at 37 degrees and counterstained with Prolong Mounting medium with DAPI. Positive foci intensity were calculated using imagine software Metamorph. EdU (5-ethynyl-2′-deoxyuridine) labeling and co-staining were carried out according to the manufacturer's instructions (#C10637, Thermo Fisher Scientific, Walthan, Mass.). γH2AX staining was performed as described above.

Cells were visualized using a Leica DM6000 B microscope with Leica Application Suite X software (Wetzlar, Germany). EdU was used at a concentration of 10 μM. EdU labeling was carried out for 1 hour in all cases, for conditions including drug treatments EdU was added during the last hour of treatment. Experiments were carried out in triplicate. Quantification of staining: Positive γH2AX staining refers to cells with over eight foci and pan-nuclear staining. For hydroxyurea (Hu) treated groups, γH2AX positive cells refers to cells where a pan-nuclear staining pattern (typically associated with replication stress) was observed.

Statistical Analysis

ANOVA with Tukey-Kramer test was used for comparing multiple treatments groups with each other. A P value<0.05 was considered significant. Repeated measure ANOVA was used for comparing multiple treatment groups measured over time. Statistical analysis of survival curves was conducted in the pancreatic cancer survival study. A log-rank (Mante-Cox) test was performed to compare mean survival among groups; p<0.05 was considered to be statistically significant.

Study Approval

All procedures involving animals and their care and use were approved by the Institutional Animal Care and Usage Committee of Stanford University.
1. Scott, A. M., Wolchok, J. D., and Old, L. J. 2012. Antibody therapy of cancer. Nat Rev Cancer 12:278-287.
2. Fabian, M. A., Biggs, W. H., 3rd, Treiber, D. K., Atteridge, C. E., Azimioara, M. D., Benedetti, M. G., Carter, T. A., Ciceri, P., Edeen, P. T., Floyd, M., et al. 2005. A small molecule-kinase interaction map for clinical kinase inhibitors. Nat Biotechnol 23:329-336.
3. Wan, L., Pantel, K., and Kang, Y. 2013. Tumor metastasis: moving new biological insights into the clinic. Nat Med 19:1450-1464.
4. Lemke, G., and Rothlin, C. V. 2008. Immunobiology of the TAM receptors. Nat Rev Immunol 8:327-336.
5. O'Bryan, J. P., Frye, R. A., Cogswell, P. C., Neubauer, A., Kitch, B., Prokop, C., Espinosa, R., 3rd, Le Beau, M. M., Earp, H. S., and Liu, E. T. 1991. axl, a transforming gene isolated from primary human myeloid leukemia cells, encodes a novel receptor tyrosine kinase. Mol Cell Biol 11:5016-5031.
6. Linger, R. M., Keating, A. K., Earp, H. S., and Graham, D. K. 2008. TAM receptor tyrosine kinases: biologic functions, signaling, and potential therapeutic targeting in human cancer. Adv Cancer Res 100:35-83.
7. Li, Y., Ye, X., Tan, C., Hongo, J. A., Zha, J., Liu, J., Kallop, D., Ludlam, M. J. C., and Pei, L. 2009. Axl as a potential therapeutic target in cancer: role of Axl in tumor growth, metastasis and angiogenesis. Oncogene 28:3442-3455.
8. Rankin, E. B., Fuh, K. C., Taylor, T. E., Krieg, A. J., Musser, M., Yuan, J., Wei, K., Kuo, C. J., Longacre, T. A., and Giaccia, A. J. 2010. AXL Is an Essential Factor and Therapeutic Target for Metastatic Ovarian Cancer. Cancer Research 70:7570-7579.
9. Vajkoczy, P., Knyazev, P., Kunkel, A., Capelle, H. H., Behrndt, S., von Tengg-Kobligk, H., Kiessling, F., Eichelsbacher, U., Essig, M., Read, T. A., et al. 2006. Dominant-negative inhibition of the Axl receptor tyrosine kinase suppresses brain tumor cell growth and invasion and prolongs survival. Proc Natl Acad Sci USA 103:5799-5804.
10. Chen, P. X., Li, Q. Y., and Yang, Z. 2013. Axl and prostasin are biomarkers for prognosis of ovarian adenocarcinoma. Ann Diagn Pathol 17:425-429.
11. Gjerdrum, C., Tiron, C., Hoiby, T., Stefansson, I., Haugen, H., Sandal, T., Collett, K., Li, S., McCormack, E., Gjertsen, B. T., et al. 2010. Axl is an essential epithelial-to-mesenchymal transition induced regulator of breast cancer metastasis and patient survival. Proc Natl Acad Sci USA 107:1124-1129.
12. Zhang, Z., Lee, J. C., Lin, L., Olivas, V., Au, V., LaFramboise, T., Abdel-Rahman, M., Wang, X., Levine, A. D., Rho, J. K., et al. 2012. Activation of the AXL kinase causes resistance to EGFR-targeted therapy in lung cancer. Nat Genet 44:852-860.
13. Hong, J., Peng, D., Chen, Z., Sehdev, V., and Belkhiri, A. 2013. ABL regulation by AXL promotes cisplatin resistance in esophageal cancer. Cancer Res 73:331-340.
14. Lu, Q., Gore, M., Zhang, Q., Camenisch, T., Boast, S., Casagranda, F., Lai, C., Skinner, M. K., Klein, R., Matsushima, G. K., et al. 1999. Tyro-3 family receptors are essential regulators of mammalian spermatogenesis. Nature 398:723-728.
15. Nagata, K., Ohashi, K., Nakano, T., Arita, H., Zong, C., Hanafusa, H., and Mizuno, K. 1996. Identification of the product of growth arrest-specific gene 6 as a common ligand for Axl, Sky, and Mer receptor tyrosine kinases. J Biol Chem 271:30022-30027.
16. Varnum, B. C., Young, C., Elliott, G., Garcia, A., Bartley, T. D., Fridell, Y. W., Hunt, R. W., Trail, G., Clogston, C., Toso, R. J., et al. 1995. Axl Receptor Tyrosine Kinase Stimulated by the Vitamin-KDependent Protein Encoded by Growth-Arrest-Specific Gene-6. Nature 373:623-626.
17. Kariolis, M. S., Miao, Y. R., Jones, D. S., 2nd, Kapur, S., Mathews, I I, Giaccia, A. J., and Cochran, J. R. 2014. An engineered Axl ‘decoy receptor’ effectively silences the Gas6-Axl signaling axis. Nat Chem Biol 10:977-983.
18. Economides, A. N., Carpenter, L. R., Rudge, J. S., Wong, V., Koehler-Stec, E. M., Hartnett, C., Pyles, E. A., Xu, X., Daly, T. J., Young, M. R., et al. 2003. Cytokine traps: multi-component, high-affinity blockers of cytokine action. Nat Med 9:47-52.
19. Kariolis, M. S., Kapur, S., and Cochran, J. R. 2013. Beyond antibodies: using biological principles to guide the development of next-generation protein therapeutics. Curr Opin Biotechnol.
20. Peppel, K., Crawford, D., and Beutler, B. 1991. A tumor necrosis factor (TNF) receptor-IgG heavy chain chimeric protein as a bivalent antagonist of TNF activity. J Exp Med 174:1483-1489.
21. Holash, J., Davis, S., Papadopoulos, N., Croll, S. D., Ho, L., Russell, M., Boland, P., Leidich, R., Hylton, D., Burova, E., et al. 2002. VEGF-Trap: a VEGF blocker with potent antitumor effects. Proc Natl Acad Sci USA 99:11393-11398.
22. Sasaki, T., Knyazev, P. G., Clout, N.J., Cheburkin, Y., Gohring, W., Ullrich, A., Timpl, R., and Hohenester, E. 2006. Structural basis for Gas6-Axl signalling. Embo Journal 25:80-87.
23. Chao, G., Lau, W. L., Hackel, B. J., Sazinsky, S. L., Lippow, S. M., and Wttrup, K. D. 2006. Isolating and engineering human antibodies using yeast surface display (vol 1, pg 755, 2006). Nature Protocols 1.
24. Park, I. K., Mishra, A., Chandler, J., Whitman, S. P., Marcucci, G., and Caligiuri, M. A. 2013. Inhibition of the receptor tyrosine kinase Axl impedes activation of the FLT3 internal tandem duplication in human acute myeloid leukemia: implications for Axl as a potential therapeutic target. Blood 121:2064-2073.
25. Thiede, C., Steudel, C., Mohr, B., Schaich, M., Schakel, U., Platzbecker, U., Wermke, M., Bornhauser, M., Ritter, M., Neubauer, A., et al. 2002. Analysis of FLT3-activating mutations in 979 patients with acute myelogenous leukemia: association with FAB subtypes and identification of subgroups with poor prognosis. Blood 99:4326-4335.
26. Holland, S. J., Pan, A., Franci, C., Hu, Y., Chang, B., Li, W., Duan, M., Torneros, A., Yu, J., Heckrodt, T. J., et al. 2010. R428, a selective small molecule inhibitor of Axl kinase, blocks tumor spread and prolongs survival in models of metastatic breast cancer. Cancer Research 70:1544-1554.
27. Kataoka, Y., Mukohara, T., Tomioka, H., Funakoshi, Y., Kiyota, N., Fujiwara, Y., Yashiro, M., Hirakawa, K., Hirai, M., and Minami, H. 2012. Foretinib (GSK1363089), a multi-kinase inhibitor of MET and VEGFRs, inhibits growth of gastric cancer cell lines by blocking inter-receptor tyrosine kinase networks. Invest New Drugs 30:1352-1360.
28. Zillhardt, M., Park, S. M., Romero, I. L., Sawada, K., Montag, A., Krausz, T., Yamada, S. D., Peter, M. E., and Lengyel, E. 2011. Foretinib (GSK1363089), an orally available multikinase inhibitor of c-Met and VEGFR-2, blocks proliferation, induces anoikis, and impairs ovarian cancer metastasis. Clin Cancer Res 17:4042-4051.
29. Ruan, G. X., and Kazlauskas, A. 2012. Axl is essential for VEGF-A-dependent activation of PI3K/Akt. EMBO J 31:1692-1703.
30. D'Arcangelo, D., Ambrosino, V., Giannuzzo, M., Gaetano, C., and Capogrossi, M. C. 2006. Axl receptor activation mediates laminar shear stress anti-apoptotic effects in human endothelial cells. Cardiovasc Res 71:754-763.
31. Rankin, E. B., Fuh, K. C., Castellini, L., Viswanathan, K., Finger, E. C., Diep, A. N., LaGory, E. L., Kariolis, M. S., Chan, A., Lindgren, D., et al. 2014. Direct regulation of GAS6/AXL signaling by HIF promotes renal metastasis through SRC and MET. Proc Natl Acad Sci USA 111:13373-13378.
32. Elkabets, M., Pazarentzos, E., Juric, D., Sheng, Q., Pelossof, R. A., Brook, S., Benzaken, A. O., Rodon, J., Morse, N., Yan, J. J., et al. 2015. AXL mediates resistance to PI3Kalpha inhibition by activating the EGFR/PKC/mTOR axis in head and neck and esophageal squamous cell carcinomas. Cancer Cell 27:533-546.
33. Bowtell, D. D., Bohm, S., Ahmed, A. A., Aspuria, P. J., Bast, R. C., Jr., Beral, V., Berek, J. S., Birrer, M. J., Blagden, S., Bookman, M. A., et al. 2015. Rethinking ovarian cancer II: reducing mortality from high-grade serous ovarian cancer. Nat Rev Cancer 15:668-679.
34. Burstyn-Cohen, T., Lew, E. D., Traves, P. G., Burrola, P. G., Hash, J. C., and Lemke, G. 2012. Genetic dissection of TAM receptor-ligand interaction in retinal pigment epithelial cell phagocytosis. Neuron 76:1123-1132.
35. Bosurgi, L., Bernink, J. H., Delgado Cuevas, V., Gagliani, N., Joannas, L., Schmid, E. T., Booth, C. J., Ghosh, S., and Rothlin, C. V. 2013. Paradoxical role of the proto-oncogene Axl and Mer receptor tyrosine kinases in colon cancer. Proc Natl Acad Sci USA 110:13091-13096.
36. Society, A. C. 2014. Cancer Facts & Figures. 2014. In Cancer Facts & Figures. 2014. Atlanta.
37. Li, D., Xie, K., Wolff, R., and Abbruzzese, J. L. 2004. Pancreatic cancer. Lancet 363:1049-1057.
38. Tseng, W. W., Winer, D., Kenkel, J. A., Choi, O., Shain, A. H., Pollack, J. R., French, R., Lowy, A. M., and Engleman, E. G. 2010. Development of an orthotopic model of invasive pancreatic cancer in an immunocompetent murine host. Clin Cancer Res 16:3684-3695.
39. Takeda Announces Phase 3 MONET-A Study Evaluating Motesanib (AMG 706) in Patients with Advanced Non-Squamous Non-Small Cell Lung Cancer Does Not Meet Primary Endpoint.
40. Wu, X., Liu, X., Koul, S., Lee, C. Y., Zhang, Z., and Halmos, B. 2014. AXL kinase as a novel target for cancer therapy. Oncotarget 5:9546-9563.
41. Ye, X., Li, Y., Stawicki, S., Couto, S., Eastham-Anderson, J., Kallop, D., Weimer, R., Wu, Y., and Pei, L. 2010. An anti-Axl monoclonal antibody attenuates xenograft tumor growth and enhances the effect of multiple anticancer therapies. Oncogene 29:5254-5264.
42. Shah, M. A., Wainberg, Z. A., Catenacci, D. V., Hochster, H. S., Ford, J., Kunz, P., Lee, F. C., Kallender, H., Cecchi, F., Rabe, D. C., et al. 2013. Phase II study evaluating 2 dosing schedules of oral foretinib (GSK1363089), cMET/VEGFR2 inhibitor, in patients with metastatic gastric cancer. PLoS One 8:e54014.
43. Emsley, P., and Cowtan, K. 2004. Coot: model-building tools for molecular graphics. Acta Crystallographica Section D-Biological Crystallography 60:2126-2132.
44. Murshudov, G. N., Vagin, A. A., and Dodson, E. J. 1997. Refinement of macromolecular structures by the maximum-likelihood method. Acta Crystallogr D Biol Crystallogr 53:240-255.
45. Schrodinger, L L C. 2010. The PyMOL Molecular Graphics System, Version 1.3r1.
46. Krissinel, E., and Henrick, K. 2007. Inference of macromolecular assemblies from crystalline state. J Mol Biol 372:774-797.
47. Aslakson, C. J., and Miller, F. R. 1992. Selective events in the metastatic process defined by analysis of the sequential dissemination of subpopulations of a mouse mammary tumor. Cancer Research 52:1399-1405.

What is claimed is:

1. A method of treating, reducing, or preventing primary tumor formation or metastasis or invasion of a tumor in a mammalian patient, the method comprising:
administering to said patient an effective dose of a soluble AXL variant polypeptide.

2. The method of claim 1, wherein the soluble AXL variant lacks the AXL transmembrane domain and has a set of amino acid modifications of the wild-type AXL sequence (SEQ ID NO:1), selected from the group consisting of:
1) Gly32Ser, Asp87Gly, Val92Ala, and Gly127Arg,
2) Glu26Gly, Val79Met, Val92Ala, and Gly127Glu; and
3) Gly32Ser, Ala72Val, Asp87Gly, Val92Ala, and Gly127Arg;
wherein said modification increases the affinity of the AXL polypeptide binding to Growth arrest-specific protein 6 (GAS6).

3. The method of claim 1, wherein the soluble AXL variant is fused to an Fc region.

4. The method of claim 1, further comprising administering one or more additional cytoreductive agents or therapy.

5. The method of claim 4, wherein the additional cytoreductive agents or therapy is selected from radiation therapy and chemotherapy.

6. The method of claim 5, wherein the additional cytoreductive agents or therapy are DNA damaging therapies.

7. The method of claim 4, wherein the combination is synergistic.

8. The method of claim 4, wherein the combination allows a reduction in the side effects of the cytoreductive therapy on the patient.

9. The method of claim 4, wherein the combination increases overall survival time of the patient.

10. The method of claim 1, wherein the cancer is acute myeloid leukemia.

11. The method of claim 1, wherein the cancer is pancreatic cancer.

12. The method of claim 1, wherein the cancer is ovarian cancer.

13. The method of claim 1, wherein the additional cytoreductive agent is gemcitabine.

14. The method of claim 1, wherein the additional cytoreductive agent is doxorubicin.


SEQUENCE LISTING (10 sequences)

SEQ ID NO: 1 - protein, 887 aa, H. sapiens
MGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 999 aa, H. sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 3 - protein, 890 aa, H. sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 4 - protein, 20 aa, H. sapiens
LRMFSGTPVIRLRFKRLQPT

SEQ ID NO: 5 - protein, 10 aa, H. sapiens
EVGRVTSSGP

SEQ ID NO: 6 - protein, 8 aa, H. sapiens
RNLVIKVN

SEQ ID NO: 7 - protein, 9 aa, H. sapiens
DAVMKIAVA

SEQ ID NO: 8 - protein, 16 aa, H. sapiens
ERGLYHLNLTVGIPFH

SEQ ID NO: 9 - protein, 17 aa, H. sapiens
WLNGEDTTIQETVVNRM

SEQ ID NO: 10 - protein, 5 aa, Artificial Sequence
  Synthetic Linker
GGGGS